(19) United States
(12) Patent Application Publication
(10) Pub. No.: US 2026/0217640 A1
(43) Pub. Date: Jul. 30, 2026

(54) SHAPE-CONTROLLED SYNTHESIS OF III-V COLLOIDAL NANOCRYSTALS

(71) Applicant: Quantum Science Ltd, Warrington, Cheshire (GB)

(72) Inventors: Joshua MEHTA, Warrington (GB); Laxmi Kishore Sagar CHILUKA, Warrington (GB)

(73) Assignee: Quantum Science Ltd, Warrington, Cheshire (GB)

(21) Appl. No.: 19/145,665

(22) PCT Filed: Jan. 5, 2024
(86) PCT No.: PCT/GB2024/050020

§ 371 Date: Jul. 3, 2025

(30) Foreign Application Priority Data
Jan. 5, 2023 (GB) 2300165.4

(51) Int. Cl. C07C 51/43; C01G 28/00; C07C 53/10; C09D 11/037

(52) U.S. Cl. CPC C07C 51/43; C01G 28/00; C07C 53/10; C09D 11/037

(57) ABSTRACT

The present invention provides a method for producing a group III-V nanocrystal composition, the method comprising forming a first mixture comprising a first indium-containing compound and/or a first gallium-containing compound, a first ligand and a first solvent, adding at least one pnictogen-containing compound to the first mixture, and heating the first mixture to a growth temperature in the range of 200° C. to 350° C.; and adding an oxide removal reagent and a composition comprising group III-V clusters to the first mixture at the growth temperature over a total growth time of at least 6 hours to provide the group III-V nanocrystal composition. The present invention also provides cuboctahedral group III-V nanocrystals, which may be formed by the method of the invention.

### FIELD OF THE INVENTION

The present invention relates in general to nanocrystals. In particular, the present invention relates to a method for producing a group III-V nanocrystal composition, the method comprising forming a first mixture comprising a first indium-containing compound and/or a first gallium-containing compound, a first ligand and a first solvent; adding at least one pnictogen-containing compound to the first mixture, and heating the first mixture to a growth temperature in the range of 200° C. to 350° C.; and adding an oxide removal reagent and a composition comprising group III-V clusters to the first mixture at the growth temperature over a total growth time of at least 6 hours to provide the group III-V nanocrystal composition. The present invention also relates to cuboctahedral group III-V nanocrystals, which may be formed by the method of the invention.

### BACKGROUND

Nanocrystals are useful in a wide range of applications, for example because their optical properties can be finely tuned to provide the desired properties. The optical properties (for example light absorption and emission characteristics) of nanocrystals can be finely tuned by controlling their size. The largest nanocrystals produce the longest wavelengths (and lowest frequencies), while the smallest nanocrystals produce shorter wavelengths (and higher frequencies). The size of the nanocrystals may be controlled by means of the method by which they are produced. This ability to finely tune the optical properties of the nanocrystals, by controlling their size, makes nanocrystals suitable for use in a wide range of applications, including, for example, photodetectors, sensors, solar cells, bio-imaging and bio-sensing, photovoltaics, displays, lighting, security and counterfeiting, batteries, wired high-speed communications, quantum dot (QD) lasers, photocatalysts, spectrometers, injectable compositions, field-effect transistors, light-emitting diodes, lasers, photonic or optical switching devices, hydrogen production and metamaterials.

Whilst lead and other heavy metal nanocrystals have been shown to exhibit good performance, their use can be problematic in some industries due to their toxicity. There is a need in the industry to provide alternative quantum dot systems that avoid the use of toxic heavy metals such as lead.

Group III-V nanocrystals represent promising candidates to replace heavy metal-based nanocrystals, having first been synthesised over a decade ago. Of particular interest are InAs and InSb nanocrystals. However, so far group III-V nanocrystals have not been able to replicate the desirable performance of lead-based nanocrystals.

InAs synthesis can generally be classified into two categories depending on the arsenic precursor used in the synthesis:
1. Tris(trimethylsilyl) arsine route-Alivisatos et al. (Appl. Phys. Lett. 1996, 69, 1432) have reported the synthesis of InAs quantum dots using the dehalosilylation reaction in presence of InCl₃+As[Si(CH₃)₃] at temperatures ranging from 240 to 265° C. with a coordinating solvent trioctylphosphine (TOP) serving as both a solvent and ligand. Recently, Bawendi et al. (Nat. Comms. 2016, 7, 12749) and Jeong et al. (Nat. Comms. 2018, 9, 1) further developed the route in the presence of indium carboxylates, octadecene and a continuous injection approach to synthesize InAs QDs. These InAs QDs have been utilized to synthesize core/shell QDs and for biological labelling and solar cells, among other uses. Recently, Sargent et al (Nano. Lett., 2021, 21, 6057) have developed InAs photodetectors at 940 nm wavelengths with an EQE of 40%.
2. Aminoarsine route-Inspired by the success of aminophosphines to develop InP QDs, Talapin et al. (Chem. Mater. 2016, 28, 6797) introduced InCl₃+aminoarsine in presence of oleylamine and a reducing agent. Bawendi et al. (J. Am. Chem. Soc., 2020, 142, 4088) have replaced In(III)Cl3 with In(I)Cl which acts as a reducing agent, eliminating the need of an external reducing agent and also acting as the indium source. Hens et al. (J. Am. Chem. Soc. 2021, 143, 4290) have developed In(P) As QDs using a similar approach replacing hydrides with aminophosphines as a reducing agent. The photodetectors developed using this approach have an EQE about 1% at 1400 nm.

However, the methods for producing group III-V nanocrystals of the prior art, including the above approaches, lead to group III-V nanocrystals with low charge transfer capabilities, which limits their use in devices such as solar cells and photodetectors.

There is therefore an unmet need for group III-V nanocrystals with improved charge transfer capabilities.

### SUMMARY OF INVENTION

According to a first aspect, the present invention provides a method for producing a group III-V nanocrystal composition, the method comprising:
a) forming a first mixture comprising a first indium-containing compound and/or a first gallium-containing compound, a first ligand and a first solvent, wherein the first ligands are C₆-C₂₄ organic compounds comprising a functional group selected from the group consisting of amino, thiol, hydroxyl and carboxylic acid;
b) adding at least one pnictogen-containing compound to the first mixture, and heating the first mixture to a growth temperature in the range of 200° C. to 350° C.;
c) adding an oxide removal reagent and a composition comprising group III-V clusters to the first mixture at the growth temperature over a total growth time of at least 6 hours to provide the group III-V nanocrystal composition.

According to a second aspect, the present invention provides a substantially cuboctahedral group III-V nanocrystal.

According to a third aspect, the present invention provides a group III-V nanocrystal composition comprising a plurality of nanocrystals according to the second aspect.

### BRIEF DESCRIPTION OF DRAWINGS

FIG. 1 is a UV-vis spectra of InAs nanocrystals with an absorbance peak at 1400 nm prepared according to the method of the first aspect of the invention (Example 2).

FIG. 2 is a UV-vis spectra of InAs nanocrystals with an absorbance peak at 1100 nm prepared according to the method of the first aspect of the invention (Example 2).

FIG. 3 shows a selection of electron micrographs of the InAs nanocrystals with an absorbance peak at 1400 nm prepared according to the method of the invention (Example 2).

FIG. 4 shows a further selection of electron micrographs of the InAs nanocrystals with an absorbance peak at 1400 nm prepared according to the method of the invention (Example 2). FIGS. 4(a) and (b) show InAs nanocrystals comprising oleic acid as a ligand compound, whereas FIGS. 4(c) and (d) show InAs nanocrystals comprising InBr3 as a ligand. FIG. 4(b) was used for the calculation of the average size of the nanocrystals having an absorption peak at 1400 nm.

FIG. 5 shows a selection of electron micrographs of the InAs nanocrystals with an absorbance peak at 1100 nm prepared according to the method of the invention (Example 2). FIG. 5(i) was used for the calculation of the average size of the nanocrystals having an absorption peak at 1100 nm.

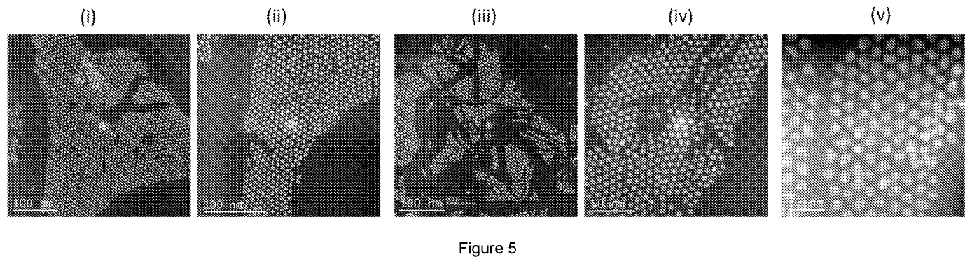

FIG. 6 shows the UV-vis spectra of InAs nanocrystals with an absorbance peak at 1100 nm and 1400 nm, both of which were prepared according to the method of the first aspect of the invention (Example 4).

FIG. 7 shows a selection of electron micrographs of InAs nanocrystals with an absorbance peak at 1100 nm and 1400 nm, both of which were prepared according to the method of the first aspect of the invention (Example 4). FIG. 7(b)(v) was used for the calculation of the average size of the nanocrystals having an absorption peak at 1100 nm.

FIG. 8 shows an electron micrograph image of InAs nanocrystals with an absorbance peak at 1400 nm prepared according to the method of the invention (Example 4). This image was used for the calculation of the average size of the nanocrystals.

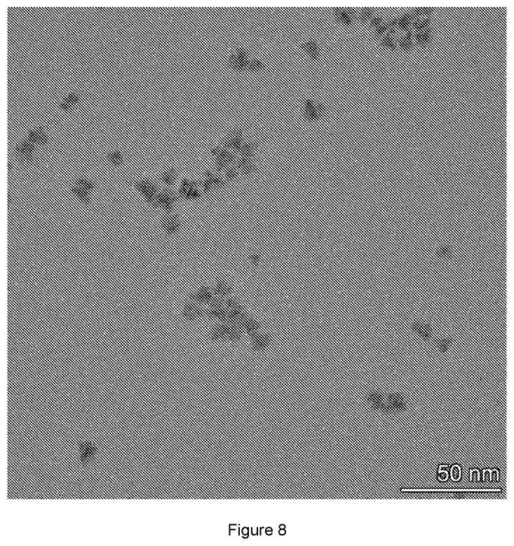

### DETAILED DESCRIPTION

When describing the aspects of the invention, the terms used are to be construed in accordance with the following definitions, unless a context dictates otherwise.

As used in the specification and the appended claims, the singular forms “a”, “an,” and “the” include both singular and plural referents unless the context clearly dictates otherwise. By way of example, “a nanocrystal” means one nanocrystal or more than one nanocrystal. By way of example, “an indium-containing compound” means one indium-containing compound or more than one indium-containing compound. References to a number when used in conjunction with comprising language include compositions comprising said number or more than said number.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

As used herein, the term “and/or,” when used in a list of two or more items, means that any one of the listed items can be employed by itself or any combination of two or more of the listed items can be employed. For example, if a list is described as comprising group A, B, and/or C, the list can comprise A alone; B alone; C alone; A and B in combination; A and C in combination, B and C in combination; or A, B, and C in combination.

As used herein, unless otherwise expressly specified, all numbers such as those expressing values, ranges, amounts of percentages may be read as if prefaced by the word “about”, even if the term does not expressly appear.

The term “about” as used herein when referring to a measurable value such as a parameter, an amount, a temporal duration, and the like, indicates that a value includes the standard deviation of error for the device or method being employed to determine the value. The term “about” is meant to encompass variations of +/−10% or less, +/−5% or less, or +/−0.1% or less of and from the specified value, insofar such variations are appropriate to perform in the disclosure. It is to be understood that the value to which the modifier “about” refers is itself also specifically disclosed.

The recitation of numerical ranges by endpoints includes all integer numbers and, where appropriate, fractions subsumed within that range (e.g. 1 to 5 can include 1, 2, 3, 4 when referring to, for example, a number of elements, and can also include 1.5, 2, 2.75 and 3.80, when referring to, for example, measurements). The recitation of end points also includes the end point values themselves (e.g. from 1.0 to 5.0 includes both 1.0 and 5.0). Any numerical range recited herein is intended to include all sub-ranges subsumed therein.

Unless otherwise defined, all terms used in the disclosure, including technical and scientific terms, have the meaning as commonly understood by one of ordinary skill in the art to which this disclosure belongs. By means of further guidance, definitions for the terms used in the description are included to better appreciate the teaching of the present disclosure. All publications referenced herein are incorporated by reference thereto.

As used herein, unless otherwise defined, the term “composition” may be open ended or closed. For example, “composition” comprises the specified material, i.e., the nanocrystals, and further unspecified material, or may consist of the specified material, i.e., to the substantial exclusion of non-specified materials.

### Method

According to a first aspect, the present invention provides a method for producing a group III-V nanocrystal composition, the method comprising:
a) forming a first mixture comprising a first indium-containing compound and/or a first gallium-containing compound, a first ligand and a first solvent, wherein the first ligands are C₆-C₂₄ organic compounds comprising a functional group selected from the group consisting of amino, thiol, hydroxyl and carboxylic acid;
b) adding at least one pnictogen-containing compound to the first mixture, and heating the first mixture to a growth temperature in the range of 200° C. to 350° C.;
c) adding an oxide removal reagent and a composition comprising group III-V clusters to the first mixture at the growth temperature over a total growth time of at least 6 hours to provide the group III-V nanocrystal composition.

Any suitable indium-containing compounds, gallium-containing compounds and pnictogen-containing compounds may be used.

As mentioned previously, heavy metal-free nanocrystals are highly desirable in the quantum dot industry due to their lower toxicity. Group III-V nanocrystals provide a promising alternative to heavy metal-based materials, but are not yet fully utilised as their performance and optical properties do not yet match those of lead-based nanocrystals.

The performance of colloidal quantum dots (CQDs) and optoelectronic devices comprising the quantum dots depends strongly on the order and interaction of the nanocrystals in solid films. It is generally understood in the art that shape control of nanocrystal synthesis may influence the order and interaction of the nanocrystals, and therefore if controlled may lead to improved optical properties of the resultant nanocrystals. For example, Pietryga et al (J. Am. Chem. Soc. 2008, 130, 14, 4879-4885) reported room temperature synthesis of large lead selenide (PbSe) nanocrystals showing cubic shape. Lee et al (Adv. Mater. 2003, 15 (5), 441) reported the shape control of different colloidal inorganic nanocrystals from nanorods to cubic shape by changing different growth parameters.

Solid films comprising colloidal quantum dots that are sufficiently surface passivated, with high packing density and coupling strength along specific facets, should show enhanced charge carrier mobility and transport.

One common approach used for improving the packing density of CQDs involves the replacement of native long-chain organic ligands on the as-synthesised quantum dots with short conductive ligands. This is generally achieved via a ligand exchange step following the initial synthesis using the native long-chain organic ligands to control nanocrystal growth. For example, Biondi et al (Adv. Mater. 2021, 2101056) exchanged the native ligands attached to PbS CQDs to increase (100):(100) facet coupling, leading to a ten-fold increase in hole mobility and faster response for devices.

However, there is a significant lack of understanding in the art regarding how to achieve beneficial shape control with group III-V nanocrystals. To the best of the inventors' knowledge, there has been no work reported in the literature on controlling the shape and the facets of short-wave infrared (SWIR) InAs and InSb quantum dots, whilst maintaining nanocrystal confinement and a sufficient degree of surface passivation. The lack of understanding on controlling InAs or InSb CQD shape to facilitate specific facet coupling in solid films may lead to the limited performance of the state-of-the-art devices. In fact, 1400 nm InAs CQDs photodiodes show only 1% EQE and high dark current as reported more recently by Hens and co-workers.

Nevertheless, the inventors of the present invention have discovered a way to control the shape of group III-V nanocrystals, leading to improved optical properties of the resultant nanocrystals.

The method of the first aspect of the invention provides a nanocrystal composition comprising substantially cuboctahedral group III-V nanocrystals by controlling the thermodynamics and reaction conditions of the nanocrystal growth. In particular, the method of the first aspect controls the nanocrystal growth by electing a suitable growth temperature in the range of 200° C. to 350° C., adding an oxide removal reagent to the first mixture, and electing a suitable total growth time of at least 6 hours. Without wishing to be bound by theory, when these conditions are met, the method of the first aspect produces substantially cuboctahedral group III-V nanocrystals.

Without wishing to be bound by theory, it is believed that the cuboctahedral shape of the group III-V nanocrystals leads to improved alignment of the facets of the nanocrystals. The better alignment of facets of the nanocrystals leads to improved charge transfer of the nanocrystals, as shown in the examples below. Furthermore, the nanocrystals produced by the method of the first aspect of the invention are stable and highly monodisperse. The method of the present invention provides a way to tune the bandgap of the nanocrystals, and delivers high crystallinity and high uniformity.

The nanocrystals produced by the method of the first aspect are very easy to phase transfer from non-polar solvents to polar solvents upon solution inorganic ligand exchange forming high quality QD inks. The resultant unique shape helps to make the nanocrystals packed into highly ordered QD films with favorable facet alignment to enhance charge transport leading to improved device performance.

The nanocrystals produced by the method of the first aspect are generally semiconductor nanocrystals, in particular quantum dots.

As used herein, the term “nanocrystal” is used to refer to a crystalline particle with at least one dimension measuring less than 100 nanometres (nm).

As used herein, the term “semiconductor nanocrystal” is used to refer to a semiconductor crystalline material exhibiting quantum confinement effects that allow it to mimic the properties of an atom, with at least one dimension measuring less than 100 nm.

As used herein, the term “quantum dot” is used to refer to a spherical semiconductor nanocrystal. Quantum dots may also be known as zero-dimensional nanocrystals.

As used herein, the term “group III-V semiconductor nanocrystal” is used to refer to a semiconductor nanocrystal comprising a group III element and a group V element.

As used herein, the term “group III element” is used to refer to an element in group 13 of the periodic table of elements. For example, a group III element may be boron, aluminium, gallium, indium, or thallium. Preferably, the group III element is indium.

As used herein, the terms “group V element” and “pnictogen” are used to refer to an element in group 15 of the periodic table of elements. For example, a group V element may be nitrogen, phosphorus, arsenic, antimony or bismuth. Preferably, the group V element is arsenic.

As used herein, the term “ligand” is used to refer to a compound capable of forming a complex with the nanocrystal by coordinating to the surface of the nanocrystal. Preferably, a plurality of ligands at least partially covers the surface of the nanocrystal. More preferably, a plurality of ligands covers the surface of the nanocrystal. A nanocrystal generally comprises a crystalline core with dimensions in the order of tens of nanometres. The nanocrystals are typically stabilised as a colloidal solution by surface capping ligands, which coordinate to the crystalline core as Lewis acidic (Z-type), Lewis basic (L-type) or anionic (X-type) species.

As used herein, the term “organic compound” is used to refer to a compound comprising carbon atoms covalently bound to other atoms. As used herein, the term “Cₓ-C_{y}” organic compound, wherein x and y are integers, is used to refer to an organic compound containing at least x and no more than y carbon atoms.

As used herein, the term “inorganic compound” is used to refer to a compound other than an organic compound.

As used herein, the terms “median particle diameter” and “average particle size” are used to refer to the Feret diameter of a particle. The Feret diameter is a measure of a particle's size along a specified direction, and is defined as the distance between two parallel tangential lines of a particle. This value can be obtained by standard imaging software or can be manually determined based on a micrograph such as those obtained by TEM.

The oxide removal reagent and the composition comprising group III-V clusters (step c) reagents) are added to the first mixture at the growth temperature over a total growth time of at least 6 hours to provide the group III-V nanocrystal composition. The oxide removal reagent may be added to the first mixture together with the composition comprising group III-V clusters or separately. The oxide removal reagent may be added to the first mixture simultaneously with the composition comprising group III-V clusters or sequentially. They may form part of the same solution prior to addition, or they may be separate solutions. Preferably, the step c) reagents are added simultaneously and separately.

The step c) reagents may be added in any suitable manner, for example by injection. The step c) reagents may be added continuously over the total growth time, or they may be added in a single injection, or a combination of these techniques. The step c) reagents may be added at the growth temperature (a temperature in the range of 200° C. to 350° C.), or they may be added at a temperature above or below the step c) reagents. Preferably, the step c) reagents are added at the growth temperature.

The first mixture may comprise an indium-containing compound and/or a gallium-containing compound. Thus, the first mixture may comprise an indium-containing compound and not comprise a gallium-containing compound. Alternatively, the first mixture may comprise a gallium-containing compound and not comprise an indium-containing compound. Alternatively, the first mixture may comprise an indium-containing compound and also comprise a gallium-containing compound. In a particularly preferred embodiment, the first mixture comprises an indium-containing compound.

Further control over the reaction may be achieved by the selection of preferred indium precursors. The preferred indium-containing compounds, and the preferred features related to the indium-containing compounds, apply to the all indium-containing compounds throughout this invention, including second indium-containing compound discussed further on. Preferably, the first mixture comprises the first indium-containing compound. Preferably, the first indium-containing compound is selected from the group consisting of indium acetate, indium triformate, indium nitrate, indium nitrate hydrate, indium sulfate, indium hydroxide, indium trifluoromethanesulfonate, indium trifluoroacetylacetonate, indium hexafluoroacetylacetonate, indium acetylacetonate, indium trifluoroacetate, indium phosphate, indium perchlorate hydrate and mixtures thereof. More preferably, the first indium-containing compound is indium acetate.

In some embodiments, the first mixture comprises the first gallium-containing compound. Where a gallium-containing compound is used, further control over the shape and reaction may be achieved by the selection of preferred gallium precursors. Preferably, the first gallium-containing compound is selected from the group consisting of gallium acetate, gallium nitrate, gallium sulfate, gallium hydroxide, gallium trifluoromethane sulfonate, gallium trifluoroacetylacetonate, gallium acetylacetonate, gallium trifluoroacetate, gallium phosphate, gallium perchlorate hydrate. Preferably, the first gallium-containing compound is gallium acetate.

In some embodiments, the first mixture comprises the first indium-containing compound and the first gallium-containing compound, and the resultant nanocrystals are ternary or quaternary. The above preferred indium-containing compounds and gallium-containing compounds apply equally to this embodiment.

The reaction takes place in a solvent in which the components of the first mixture are soluble. The indium-containing compounds, gallium-containing compounds and pnictogen-containing compounds are generally soluble in both polar solvents and non-poalr solvents. Generally, as the initial synthesis uses long-chain organic ligands, a non-polar solvent is used. Preferably, the first solvent is therefore a non-polar solvent. Preferably, the first solvent has a relative permittivity at 20° C. of less than 3, more preferably less than 2.5. As used herein, the term “relative permittivity” is used to refer to the ratio of the permittivity of a substance to the permittivity of a vacuum; it is a dimensionless number. As defined in Permittivity (Dielectric Constant) of Liquids, by Christian Wohlfarth, the permittivity of a substance (often called the dielectric constant) is the ratio of the electric displacement D to the electric field strength E when an external field is applied to the substance. The relative permittivity is measured with a BI-870 Dielectric Constant Meter available from Brookhaven Instruments, using the sensitivity range of 1 to 200. The instrument may be calibrated with a liquid of known relative permittivity. Polar solvents generally have higher relative permittivities, whilst non-polar solvents generally have lower relative permittivities.

Preferably, the first solvent is selected from the group consisting of hydrocarbons of formula CₙH₂ₙ₊₂, alkylamines of formula CₙH₂ₙ₊₁NH₂, octadecene, oleylamine, octylamine, butylamine, dioctylamine, trioctylamine, heptadecane, hexadecane, oleic acid, tertiary phosphines and secondary phosphines, wherein n is in the range of 6 to 20.

The components in the first mixture may be combined in any suitable amounts and in any suitable order, and these suitable amounts would be obvious to the skilled person based on common general knowledge. Preferably, the molar ratio of the at least one pnictogen-containing compound to the first indium-containing compound and the first gallium-containing compound is in the range of 0.5:1 to 1:0.5, preferably in the range of 0.5:1 to 1:1, preferably in the range of 0.6:1 to 1:1. The molar ratio of the at least one pnictogen-containing compound to the first indium-containing compound and the first gallium-containing compound (MR1) is defined as the total moles of all pnictogen-containing compounds in the first mixture to the sum of the total moles of all indium-containing compounds and the total moles of all gallium-containing compounds in the first mixture, as illustrated in the below equation:

$$MR 1 = \frac{total moles of all pnictogen - containing compounds}{\begin{matrix}total moles of first indium - containing compound + \\ total moles of first gallium - containing compound\end{matrix}}$$

The indium-containing compound and the first gallium-containing compound are each group III precursors, meaning that they are compounds containing a group Ill element, as defined above. The ratio (MR2) of group V elements (pnictogens) in the first mixture to group III elements is generally the same as MR1. MR2 is preferably in the range of 0.5:1 to 1:0.5, preferably in the range of 0.5:1 to 1:1, preferably in the range of 0.6:1 to 1:1. The group V elements may be present in the first mixture as part of a pnictogen-containing compound, and the group III elements may be present in the first mixture as part of a group III precursor.

In an embodiment, step b) comprises adding one pnictogen-containing compound, wherein the pnictogen-containing compound is selected from the group consisting of a phosphorus-containing compound, an arsenic-containing compound and an antimony-containing compound. By adding one pnictogen-containing compound, the resultant nanocrystal will comprise one pnictogen in the crystalline core. Therefore, this embodiment may produce InAs nanocrystals, InSb nanocrystals, InP nanocrystals, InGaAs nanocrystals, InGaSb nanocrystals, InGaP nanocrystals, GaAs nanocrystals, GaSb nanocrystals and GaP nanocrystals.

In an embodiment, step b) comprises adding two pnictogen-containing compounds, wherein a first pnictogen-containing compound is an arsenic-containing compound, and wherein a second pnictogen-containing compound is a phosphorus-containing compound or an antimony-containing compound. By adding two pnictogen-containing compounds, the resultant nanocrystal will comprise two pnictogens in the crystalline core. Therefore, this embodiment may produce InAsSb nanocrystals, InAsP nanocrystals, InGaAsSb nanocrystals and InGaAsP nanocrystals.

Preferably, the arsenic-containing compound is selected from the group consisting of an tris(trialkylsilyl) arsine, wherein the alkyl is methyl, ethyl, propyl, butyl or substituted, tris(trimethylgermyl) arsine, tris(triphenylsilyl) arsine, triphenyl arsine, aminoarsine (R₂N)₃As, wherein R is alkyl or aryl, arsenic halides, alkyl or aryl arsenous acids and mixtures thereof. Preferably, the arsenic-containing compound is selected from the group consisting of tris(trimethylsilyl) arsine, tris(trimethylgermyl) arsine, tris(dimethylamino) arsine and mixtures thereof. A particularly preferred arsenic-containing compound is tris(trimethylsilyl) arsine.

Preferably, the antimony-containing compound is selected from the group consisting of tris(trialkylsilyl) antimony, wherein the alkyl is methyl, ethyl, propyl, butyl or substituted, tris(trimethylgermyl) antimony and aminoantimony (R₂N)₃Sb, wherein R is alkyl or aryl, antimony halides, antimony dialkaylamides, dialkylsilylamides, antimony hydrides, antimony aziridinides, antimony thiolates, antimony carbamates, antimony guanidinates and mixtures thereof. Preferably, the antimony-containing compound is selected from the group consisting of tris(trimethylsilyl) antimony, tris(trimethylgermyl) antimony, tris(dimethylamino) antimony and mixtures thereof.

Preferably, the phosphorus-containing compound is selected from the group consisting of an tris(trialkylsilyl) phosphine, wherein the alkyl is methyl, ethyl, propyl, butyl or substituted, tris(trimethylgermyl) phosphine, tris(triphenylsilyl) phosphine, triphenyl phosphine, aminophosphine (R₂N)₃P, wherein R is alkyl or aryl, phosphonic halides, alkyl or aryl phosphonic acids and mixtures thereof. Preferably, the phosphorus-containing compound is selected from the group consisting of tris(trimethylsilyl) phosphine, tris(trimethylgermyl) phosphine, tris(dimethylamino)phosphine and mixtures thereof. A particularly preferred phosphorus-containing compound is tris(trimethylsilyl) phosphine.

The ligands of the present invention (e.g. the first ligand and the second ligand) are generally provided as pluralities of ligands. The term “a first ligand” is used to refer to the ligand component of the first mixture, and it would be readily understood that the first ligand components will comprise a plurality of ligands. Likewise, this applies to the second ligand in the second mixture, as discussed below.

The first ligands are C₆-C₂₄ organic compounds comprising a functional group selected from the group consisting of amino, thiol, hydroxyl and carboxylic acid. The first ligands may be any such compounds capable of forming a complex with nanocrystals by coordinating to a surface of the nanocrystal.

The molar ratio of the first ligand to the first indium-containing compound and/or gallium-containing compound may be any suitable molar ratio, and these suitable values will be obvious to the person skilled in the art in view of the common general knowledge. The molar ratio of the first ligand to the first indium-containing compound and/or gallium-containing compound may be sufficient for the ligands to coordinate to the crystalline core of the developing nanocrystals to stabilise the growth of the nanocrystals. Preferably, the molar ratio of the first ligand to the first indium-containing compound and/or gallium-containing compound is at least 2:1, preferably at least 2.5:1.

Preferably, the first ligand is selected from the group consisting of alkylamines of formula CₙH₂ₙ₊₁NH₂, alkyl carboxylic acids of formula CₙH₂ₙ₊₁COOH, alkyl phosphonic acids of formula CₙH₂ₙ₊₁PO₃H₂, aromatic phosphonic acids, alkyl or aryl thiols, alkyl or aryl arsenous acids, and mixtures thereof, wherein n is in the range of 6 to 20. Preferably, the first ligand is selected from the group consisting of aminobenzoic acids, dicarboxylic acids, aminoalkylcarboxylic acids, mercaptopropionic acid, mercaptobenzoic acid, thioalkanes, dithioalkanes, thiocarboxylic acids, thioglycolic acid, poly(ethylene glycol), poly(ethylene glycol) bis(3-aminopropyl) terminated, didodecyldimethylammonium bromide, n-dodecylammonium bromide, dodecyltrimethylammonium bromide, dimercaptosuccinic acid, oleic acid, oleylamine, bis(diphenylphosphino) methane and alkylamines. Preferably, the first ligand is selected from the group consisting of mercaptopropionic acid, mercaptobenzoic acid, thioglycolic acid, poly(ethylene glycol), poly(ethylene glycol) bis(3-aminopropyl) terminated, didodecyldimethylammonium bromide, n-dodecylammonium bromide, dodecyltrimethylammonium bromide, dimercaptosuccinic acid, oleic acid, oleylamine, bis(diphenylphosphino) methane.

The growth temperature may be any temperature at which the nanocrystals grow. Maintaining the mixture at the growth temperature may comprise maintaining the temperature of the mixture at a specific suitable temperature. However, it is to be understood that the temperature may vary during the total growth time, and during the first and second growth times as discussed below. Therefore, maintaining the first mixture at the growth temperature may comprise maintaining the temperature of the first mixture within the range of suitable growth temperatures, for example within the range of 200° C. to 350° C.

Preferably, the growth temperature is in the range of 220° C. to 340° C., preferably in the range of 240° C. to 330° C., preferably in the range of 260° C. to 320° C., preferably in the range of 270° C. to 300° C.

The growth temperature may be varied to produce nanocrystals of different sizes. For example, a lower growth temperature (e.g. 200° C. to 250° C.) may lead to smaller nanocrystals, whereas a higher growth temperature (e.g. above 270° C.) may lead to larger nanocrystals. Therefore, if smaller nanocrystals are desired, a lower growth temperature may be preferred.

A growth time is the length of time that the first mixture is at a suitable growth temperature, for example the growth time may be the length of time that the first mixture is at a temperature in the range of 200° C. to 350° C. The total growth time is the total length of time that the first mixture is at a growth temperature during the synthesis. The total growth time may be one period of time without any breaks, i.e. without the temperature of the first mixture being above or below the range of suitable growth temperatures. Alternatively, the total growth time may be split into segments, for example a first growth time and a second growth time, between which the temperature of the first mixture may be below or above the range of suitable growth temperatures. For example, the first mixture may be maintained at a suitable growth temperature for a first growth time of 4 hours, cooled, and then heated up again to a suitable growth temperature for a second growth time of 8 hours. In this example, the total growth time will be 12 hours.

Preferably, the total growth time is in the range of 6 hours to 30 hours, preferably in the range of 8 hours to 26, preferably in the range of 8 hours to 24 hours, preferably in the range of 9 hours to 23 hours, preferably in the range of 10 hours to 22 hours.

Contrary to what has been thought in previous literature, the inventors have found that the slow growth of the nanocrystals, with long total growth times as required by the method of the first aspect of the present invention, allows more thermodynamic control of the nanocrystal facets. The longer total growth times generally result in cuboctahedral nanocrystals.

The total growth time may be varied to tune the bandgap of the resultant nanocrystals. For example, a shorter total growth time (e.g. in the range of 6 hours to 10 hours) may lead to InAs nanocrystals with an absorption peak of around 1400 nm, while a longer total growth time (e.g. more than 12 hours) may lead to InAs nanocrystals with an absorption peak of around 1600 nm. This tunability is extremely advantageous and allows the production of a wide variety of nanocrystals for a wide variety of purposes.

Where a smaller bandgap is desired, the total growth time is preferably in the range of 8 hours to 19 hours, preferably in the range of 9 hours to 18 hours, preferably in the range of 10 hours to 17 hours, preferably in the range of 11 hours to 16 hours.

Where a larger bandgap is desired, the total growth time is preferably in the range of 14 hours to 28 hours, preferably in the range of 15 hours to 26 hours, preferably in the range of 16 hours to 24 hours, preferably in the range of 17 hours to 22 hours.

Preferably, the nanocrystal composition comprises group III-V nanocrystals, wherein the nanocrystals comprise a crystalline core and a plurality of first ligands coordinated to a surface of the crystalline core. The crystalline core generally comprises the indium and/or gallium that is introduced by the indium-containing compounds and/or the gallium containing compounds. The crystalline core generally comprises the pnictogen(s) introduced by the pnictogen-containing compounds. Therefore, a large variety of different nanocrystal systems may be produced by the method of the first aspect.

As the nanocrystals grow, the first ligands coordinate to the surface of the crystalline core of the nanocrystals, thereby stabilising the nanocrystals and aiding their growth.

An oxide removal agent is used in the method of the first aspect to at least partially remove any surface oxidation that may occur on the surface of the crystalline core of the nanocrystals as they grow. It has been hypothesised, for example by Chaudret et al. (Journal of American Chemical Society, 2010, 18147-18157, 132 (51)), that surfaces of the crystalline core may become oxidised during the nanocrystal synthesis. Without wishing to be bound by theory, it is believed that the layers of oxidised material surrounding the nanocrystal created by the surface oxidation prevent and/or hamper further growth of the nanocrystals. Surface oxidation is therefore undesirable.

The oxide removal agent of the present invention may be any suitable compound or composition, and these suitable oxide removal agents will be recognised by those skilled in the art in view of the common general knowledge. Preferably, the oxide removal reagent is a compound capable of reacting with oxides on the surface of the crystalline core to remove the oxides from the surface of the crystalline core. Preferably, the oxide removal agent is an organic acid, an organic alkali or a metal halide. Preferably, the oxide removal reagent is selected from the group consisting of hydrochloric acid, hydrobromic acid, hydroiodic acid, hydrofluoric acid, hypo phosphorus acid, phosphoric acid, ammonium hydroxide, potassium hydroxide, sodium hydroxide, zinc halides, indium halides and metal halides, wherein the metal is bismuth, lead or cadmium. In a particularly preferred embodiment, the oxide removal reagent is hydrobromic acid.

The oxide removal agent is preferably added in small quantities, for example 10 ppm. Preferably, the molar ratio of the oxide removal reagent to the sum of the first indium-containing compound and/or first gallium-containing compound is in the range of 1:100 to 1:1,000,000, preferably in the range of 1:1000 to 1:100,000, more preferably in the range of 1:000 to 1:50,000. Preferably, the amount of oxide removal reagent in the first mixture is from 0.0001 mmol to 0.005 mmol, preferably in the range of 0.00025 mmol to 0.0025 mmol. This may correspond to a concentration in the first mixture immediately after addition of the oxide removal reagent in the range of 5 μM to 1000 μM, preferably in the range of 50 UM to 500 UM.

Preferably, steps a), b) and c) take place under inert conditions. For example, steps a), b) and c) may take place under nitrogen, argon, or another inert gas.

In a preferred embodiment, step a) further comprises degassing the first mixture under vacuum (~0.1 mbar) for a degassing time in the range of 5 to 180 minutes at a degassing temperature in the range of 50° C. to 150° C. Preferably, the degassing time is in the range of 20 minutes to 160 minutes, preferably in the range of 40 minutes to 140 minutes, preferably in the range of 60 minutes to 120 minutes, preferably in the range of 80 minutes to 100 minutes. Preferably, the degassing temperature is in the range of 70° C. to 150° C., preferably in the range of 80° C. to 140° C., preferably in the range of 90° C. to 130° C., preferably in the range of 100° C. to 120° C.

Preferably, step a) further comprises heating the first mixture to a first temperature in the range of 50° C. to 150° C. Preferably, the first temperature is in the range of 60° C. to 140° C., preferably in the range of 70° C. to 130° C., preferably in the range of 80° C. to 120° C., preferably in the range of 90° C. to 110° C.

Where degassing and heating the first mixture to a first temperature, degassing takes place before heating the first mixture to the first temperature.

Preferably, step c) comprises:
i) adding a first oxide removal reagent and the composition comprising group III-V clusters to the first mixture at the growth temperature over a first growth time in the range of 2 hours to 8 hours to provide a plurality of nanocrystals in the first mixture;
ii) allowing the first mixture to cool;
iii) heating the first mixture to the growth temperature;
iv) adding a third oxide removal reagent and the composition comprising group III-V clusters to the first mixture over a second growth time in the range of 6 hours to 20 hours at the growth temperature to provide the group III-V nanocrystal composition.

When step c) comprises steps i) to iv) as above, the method of the first aspect essentially involves the following steps:
forming a first mixture comprising a first indium-containing compound and/or a first gallium-containing compound, a first ligand and a first solvent, wherein the first ligands are C₆-C₂₄ organic compounds comprising a functional group selected from the group consisting of amino, thiol, hydroxyl and carboxylic acid;
adding at least one pnictogen-containing compound to the first mixture, and heating the first mixture to a growth temperature in the range of 200° C. to 350° C.;
adding a first oxide removal reagent and the composition comprising group III-V clusters to the first mixture at the growth temperature over a first growth time in the range of 2 hours to 8 hours to provide a plurality of nanocrystals in the first mixture;
allowing the first mixture to cool;
heating the first mixture to the growth temperature;
adding a third oxide removal reagent and the composition comprising group III-V clusters to the first mixture over a second growth time in the range of 6 hours to 20 hours at the growth temperature to provide the group III-V nanocrystal composition.

Preferably, step iii) further comprises adding a second oxide removal reagent to the first mixture before heating the first mixture to the growth temperature.

Preferably, step i) further comprises heating the first mixture to a third temperature in the range of 50° C. to 150° C., preferably the range of 60° C. to 140° C., preferably the range of 70° C. to 130° C., preferably the range of 80° C. to 120° C., preferably the range of 90° C. to 110° C. The second oxide removal reagent is preferably added when the first mixture is at the third temperature.

The plurality of nanocrystals produced by step i) generally exhibit a lower absorption peak. For example, for a plurality of InAs nanocrystals produced by step i), the absorption peak may be around 1100 nm. This absorption peak then increases over the total growth time.

Preferably, the first growth time is in the range of 2.5 hours to 7 hours, preferably in the range of 3 hours to 6 hours, preferably in the range of 3.5 hours to 5.5 hours, preferably in the range of 4 hours to 5 hours.

Preferably, the second growth time is in the range of 6 hours to 18 hours, preferably in the range of 6.5 hours to 16 hours, preferably in the range of 7 hours to 15 hours.

As explained above, the total growth time here would be the sum of the first growth time and the second growth time.

Preferably, the nanocrystal composition comprises group III-V nanocrystals, wherein the nanocrystals comprise a crystalline core and a plurality of first ligands coordinated to a surface of the crystalline core, and wherein the first oxide removal reagent, the second oxide removal reagent and the third oxide removal reagent are each independently a compound capable of reacting with oxides on the surface of the crystalline core to remove the oxides from the surface of the crystalline core.

The first oxide removal reagent, the second oxide removal reagent and the third oxide removal reagent are each independently selected with the same preferences as explained above with regard to the oxide removal agent in step c).

In an embodiment, the first oxide removal reagent, the second oxide removal reagent and the third oxide removal reagent are the same compound or different compounds. Preferably, the first oxide removal reagent, the second oxide removal reagent and the third oxide removal reagent are the same compound. Preferably, the first oxide removal reagent, the second oxide removal reagent and the third oxide removal reagent are hydrobromic acid.

Preferably, the first mixture comprises a compound selected from the group consisting of secondary amines, zinc carboxylates, and mixtures thereof, preferably wherein the compound is dioctyl amine. This compound is used to further enhance the shape control of the nanocrystals by complexing to the pnictogen-containing compound. In this way, it is possible to reduce the reactivity of the pnictogen-containing compound, which leads to better control over the size and the size distribution of the nanocrystals. Preferably, this compound is used when the pnictogen-containing compound is tris(trimethylsilyl) arsine or tris(trimethylsilyl) antimony, as these compounds are especially reactive.

The method of the first aspect involves the addition of group III-V clusters into the first mixture. A group III-V cluster, as defined herein, is an amorphous particle comprising a group III element and a group V element, wherein the diameter of the particle is less than 2 nm, as measured by TEM. Occasionally, these particles are referred to as magic-sized particles (MSCs). The composition comprising group III-V clusters used in the method of the first aspect of the present invention may be prepared beforehand or may be prepared as part of the method of the first aspect of the invention.

In an embodiment, the method further comprises preparing the composition comprising group III-V clusters, comprising:
A) adding a second indium-containing compound and/or a second gallium-containing compound, and a second ligand to a second solvent to form a second mixture, wherein the second ligand is a C₆-C₂₄ organic compound comprising a functional group selected from the group consisting of amino, thiol, hydroxyl and carboxylic acid;
B) adding at least one pnictogen-containing compound to the second mixture under constant agitation to provide the group III-V clusters.

Preferably, the group III-V clusters comprise phosphorus, arsenic, antimony or mixtures thereof. Preferably, the group III-V clusters are amorphous clusters exhibiting an absorption peak in the range of 250 nm to 1200 nm, preferably in the range of 300 nm to 700 nm. Typically, InP clusters show characteristic absorption peak features around 330-400 nm. InAs clusters show these absorption features from 370-570 nm. InSb clusters show characteristic absorption peak features around 380-650 nm. InGaAs clusters show characteristic absorption peak features around 300-400 nm.

The second mixture generally corresponds to the first mixture. For example, if the first mixture comprises a first indium-containing compound, the second mixture should generally comprise the second indium-containing compound.

Furthermore, the preferences of the first indium-containing compound and the second indium-containing compound are the same, i.e. the second indium-containing compound is preferably any of the indium-containing compounds specified above in relation to the first indium-containing compound, in particular indium acetate. The first indium-containing compound and the second indium-containing compound may be the same or they may be different. Preferably, the first indium-containing compound and the second indium-containing compound are the same.

Preferably, the second mixture comprises the second indium-containing compound.

In an embodiment, the second mixture comprises the second gallium-containing compound. Furthermore, the preferences of the first gallium-containing compound and the second gallium-containing compound are the same, i.e. the second gallium-containing compound is preferably any of the gallium-containing compounds specified above in relation to the first gallium-containing compound, in particular gallium acetate. The first gallium-containing compound and the second gallium-containing compound may be the same or they may be different. Preferably, the first gallium-containing compound and the second gallium-containing compound are the same.

In an embodiment, the second mixture comprises the second indium-containing compound and the second gallium-containing compound.

The preferences of the first solvent and the second solvent are the same, i.e. the second solvent is preferably any of the solvents specified above in relation to the first solvent. The first solvent and the second solvent may be the same or they may be different. Preferably, the first solvent and the second solvent are the same. Preferably, the second solvent is a non-polar solvent. Preferably, the second solvent has a relative permittivity at 20° C. of less than 3, more preferably less than 2.5. Preferably, the second solvent is selected from the group consisting of hydrocarbons of formula CₙH₂ₙ₊₂, alkylamines of formula CₙH₂ₙ₊₁NH₂, octadecene, oleylamine, octylamine, butylamine, dioctylamine, trioctylamine, heptadecane, hexadecane, oleic acid, tertiary phosphines and secondary phosphines, wherein n is in the range of 6 to 20. Preferably, the second solvent is the same as the first solvent.

The molar ratios as specified above in relation to the first indium-containing compound and/or the first gallium-containing compound apply equally to the preferred ratios in the method for producing the group III-V clusters.

The components in the second mixture may be combined in any suitable amounts and in any suitable order, and these suitable amounts would be obvious to the skilled person based on common general knowledge. Preferably, the molar ratio of the at least one pnictogen-containing compound to the second indium-containing compound and the second gallium-containing compound is in the range of 0.5:1 to 1:0.5, preferably in the range of 0.5:1 to 1:1, preferably in the range of 0.6:1 to 1:1. The molar ratio of the at least one pnictogen-containing compound to the second indium-containing compound and the second gallium-containing compound (MR3) is defined as the total moles of all pnictogen-containing compounds in the second mixture to the sum of the total moles of all indium-containing compounds and the total moles of all gallium-containing compounds in the first mixture, as illustrated in the below equation:

$$MR 3 = \frac{total moles of all pnictogen - containing compounds}{\begin{matrix}total moles of second indium - containing compound + \\ total moles of second gallium - containing compound\end{matrix}}$$

The second indium-containing compound and the second gallium-containing compound are each group Ill precursors. The ratio (MR4) of group V elements (pnictogens) to group III elements in the second mixture is generally the same as MR3. MR4 is preferably in the range of 0.5:1 to 1:0.5, preferably in the range of 0.5:1 to 1:1, preferably in the range of 0.6:1 to 1:1. The group V elements may be present in the second mixture as part of a pictogen-containing compound, and the group III elements may be present in the second mixture as part of a group III precursor.

Preferably, the molar ratio of the at least one pnictogen-containing compound to the second indium-containing compound and the second gallium-containing compound is in the range of 0.5:1 to 1:0.5, preferably in the range of 0.5:1 to 1:1, preferably in the range of 0.6:1 to 1:1.

In an embodiment, step B) comprises adding one pnictogen-containing compound, wherein the pnictogen-containing compound is selected from the group consisting of a phosphorus-containing compound, an arsenic-containing compound and an antimony-containing compound.

In an embodiment, step B) comprises adding two pnictogen-containing compounds, wherein a first pnictogen-containing compound is an arsenic-containing compound, and wherein a second pnictogen-containing compound is a phosphorus-containing compound or an antimony-containing compound.

The preferences of the first phosphorus-containing compound and the second phosphorus-containing compound are the same, i.e. the second phosphorus-containing compound is preferably any of the phosphorus-containing compounds specified above in relation to the first phosphorus-containing compound. The first phosphorus-containing compound and the second phosphorus-containing compound may be the same or they may be different. Preferably, the first phosphorus-containing compound and the second phosphorus-containing compound are the same.

The preferences of the first arsenic-containing compound and the second arsenic-containing compound are the same, i.e. the second arsenic-containing compound is preferably any of the arsenic-containing compounds specified above in relation to the first arsenic-containing compound. The first arsenic-containing compound and the second arsenic-containing compound may be the same or they may be different. Preferably, the first arsenic-containing compound and the second arsenic-containing compound are the same.

The preferences of the first antimony-containing compound and the second antimony-containing compound are the same, i.e. the second antimony-containing compound is preferably any of the antimony-containing compounds specified above in relation to the first antimony-containing compound. The first antimony-containing compound and the second antimony-containing compound may be the same or they may be different. Preferably, the first antimony-containing compound and the second antimony-containing compound are the same.

As above, the molar ratio of the second ligand to the second indium-containing compound may be any suitable ratio, and is preferably at least 2:1, preferably at least 2.5:1.

The preferences of the first ligand and the second ligand are the same, i.e. the second ligand is preferably any of the ligands specified above in relation to the first ligand. The first ligand and the second ligand may be the same or they may be different. Preferably, the first ligand and the second ligand are the same.

A group III-V nanocrystal composition obtainable by the method of the first aspect of the invention. The group III-V nanocrystal composition obtainable by the method of the first aspect of the invention would be easily distinguished from the nanocrystal compositions prepared by methods of the prior art as the nanocrystals produced by the method of the present invention are cuboctahedral.

### Product

According to a second aspect, the present invention provides a substantially cuboctahedral group III-V nanocrystal. Preferably, the nanocrystal is cuboctahedral. As far as the present inventors are aware, there has not been a disclosure of cuboctaheral group III-V nanocrystals, and the shape leads to improved optoelectronic properties as discussed above.

As used herein, the term “cuboctahedral” is used to refer to the supramolecular structure of a crystalline material, wherein the 3D shape of the material resembles a cuboctahedron. A cuboctahedron is a polyhedron with 8 triangular faces and 6 square faces. A cuboctahedron has 12 identical vertices, with 2 triangles and 2 squares meeting at each, and 24 identical edges, each separating a triangle from a square. The shape and structure are well understood by those in the art, and may be analysed via TEM. Typically, the shape of the nanocrystals is determined from the TEM and HR TEM images, either with the human eye or via image analysis software (for example, imageJ) which requires a user input.

High resolution TEM (HRTEM) may be used to image the nanocrystals and determine their shape. Fast Fourier Transform (FFT) analysis may be performed on the HRTEM micrographs. If nanocrystals are cuboctahedral, the nanocrystals will often appear in the TEM images viewed along the zone axis with six bounding facets (four {111} faces and two {002} faces). These characteristics correspond to a single-crystalline fcc nanoparticle with a cuboctahedron shape. HR TEM images of single particles bring more details in determining the shape accurately

In a cuboctahedral group III-V nanocrystal, the atoms forming the cuboctahedron are generally the group III elements and the group V elements. Preferably, the nanocrystal comprises a crystalline core comprising indium and/or gallium, and a pnictogen. Preferably, the pnictogen is phosphorus, arsenic, antimony, or a mixture thereof. Preferably, the pnictogen is arsenic, antimony, or a mixture thereof.

In an embodiment, a plurality of ligands are coordinated to a surface of the crystalline core of the nanocrystals. Preferably, the ligands are organic compounds, inorganic compounds, or a mixture thereof. The preferences of the ligands of the second aspect and the ligands of the first aspect are the same, i.e. the ligands of the second aspect are preferably any of the ligands specified above in relation to the first aspect.

The absorption peaks exhibited by the nanocrystals vary significantly depending on the composition of the crystalline core of the nanocrystals. In general, the nanocrystal composition exhibits an absorption peak in the range of 500 nm to 3000 nm. The absorption peak may be tuned according to the desired function of the nanocrystals to anywhere in the range of known confinement ranges for each nanocrystal system. For example, the absorption peak position of InAs could be in the range of 500 nm to 1700 nm. For InAs/Sb nanocrystals, the absorption peak could be in the range of 700 nm to 2200 nm, and may also be varied by changing the antimony composition, reaction conditions, temperature and growth time, for example. For InSb, the absorption peak could be in the range of 700 nm to 3000 nm.

Where the crystalline core consists of indium and arsenic, the absorption peak is preferably in the range of 1300 nm to 1700 nm, preferably in the range of 1320 nm to 1680 nm, preferably in the range of 1340 nm to 1660 nm, preferably in the range of 1360 nm to 1640 nm, preferably in the range of 1380 nm to 1620 nm, preferably in the range of 1400 nm to 1600 nm. Preferably, the InAs nanocrystal composition exhibits an emission peak in the range of 1300 nm to 1900 nm, preferably in the range of 1320 nm to 1880 nm, preferably in the range of 1340 nm to 1860 nm, preferably in the range of 1360 nm to 1840 nm, preferably in the range of 1380 nm to 1820 nm, preferably in the range of 1400 nm to 1800 nm.

It is an aim in the industry to grow the size of InAs QDs in order to push their absorption further into the SWIR region. An absorption peak beyond the ‘eye-safe threshold’ of 1350 nm is of particular interest for photosensing applications. However, despite advances in the synthesis of colloidal InPn QDs, previous attempts in the prior art to push the lowest energy electronic transition (LEET) beyond 1400 nm have resulted in a concomitant loss of synthetic control and severe peak broadening.

Extending the first excitonic transition energy beyond 1400 nm whilst maintaining a homogeneous size distribution remains a significant challenge. Key optical metrics, such as the peak to valley (P/V) ratio and the exciton full-width-at-half-maximum (FWHM) tend to decrease and broaden, respectively, as the QDs are grown beyond ~7 nm. Whilst this can partially be explained by the morphology-dependent absorbance of InPn QDs, which changes as the QDs grow, generally a loss of synthetic control leads to non-specific absorbance profiles.

The present invention provides the ability to push the lowest energy electronic transition (LEET) beyond 1400 nm and up to higher than 1600 nm, as shown in the examples below. This is an additional advantage of the present invention.

For other systems, such as InAs/Sb and InSb, the absorption peak may be pushed higher than for InAs nanocrystals. For InAs/Sb systems, the absorption peak is preferably in the range of 700 nm to 2200 nm, preferably in the range of 1000 nm to 2000 nm, preferably in the range of 1200 nm to 1800 nm. For InSb systems, the absorption peak is preferably in the range of 700 nm to 3000 nm, preferably in the range of 1000 nm to 2500 nm, preferably in the range of 1300 nm to 2200 nm.

According to a third aspect, the present invention provides a group III-V nanocrystal composition comprising a plurality of nanocrystals according to the second aspect.

The nanocrystals present in the nanocrystal composition of the third aspect may additionally comprise nanocrystals that are not cuboctahedral. Preferably, at least 50% of the nanocrystals present in the nanocrystal composition are cuboctaheral, preferably at least 60%, preferably at least 70%, preferably at least 80%, preferably 90%. This may be determined by TEM.

Preferably, the nanocrystals have a median particle diameter in the range of 2 nm to 20 nm, preferably 2.5 nm to 15 nm, preferably 3 nm to 12 nm, preferably 4 nm to 10 nm, as measured by TEM.

Preferably, the nanocrystal composition exhibits absorption full width at half maximum (FWHM) values of less than 250 nm, preferably less than 225 nm, preferably less than 200 nm, preferably less than 175 nm.

Preferably, the nanocrystal composition exhibits emission full width at half maximum (FWHM) values of less than 250 nm, preferably less than 225 nm, preferably less than 200 nm, preferably less than 175 nm.

Preferably, the nanocrystal composition exhibits a peak to valley ratio in the range of 1 to 4, preferably in the range of 1 to 3.5, preferably in the range of 1 to 3, preferably in the range of 1.1 to 2.5, preferably in the range of 1.2 to 2.5.

Preferably, the nanocrystals exhibit a relative size dispersion of less than 25%, preferably less than 15%, preferably less than 10%, preferably in the range of 1% to 25%, preferably in the range of 1% to 20%, preferably in the range of 2% to 15%, preferably in the range of 3% to 10%.

The relative size dispersion is a measure of the variance of the nanocrystal particle size. It is determined by measuring the particle sizes of a particular batch of nanoparticles, and determining the variance to the mean size. This can be expressed as a particular average size, x, plus or minus the range of particle size. The relative size dispersion is measured by TEM.

According to a fourth aspect, the present invention provides an ink comprising the group III-V nanocrystal composition of the third aspect. Formation of the ink generally comprises a ligand exchange step whereby the long chain organic ligands are replaced by conducting ligands, for example inorganic ligands or short chain organic ligands. The nanocrystals are dissolved in a polar solvent, optionally after being washed with acetone. The polar solvent may be selected from the group consisting of 2,6-difluoropyridine, propylene carbonate, dimethylformamide and combinations thereof.

According to a fifth aspect, the present invention provides a film comprising the group III-V nanocrystal composition of the third aspect.

According to a sixth aspect, the present invention provides a device selected from the group consisting of IR sensor, photodetector, sensor, solar cell, a bio-imaging or bio-sensing composition, photovoltaic system, display, battery, laser, photocatalyst, spectrometer, injectable composition, field-effect transistor, light-emitting diode, photonic or optical switching device or metamaterial, fiber amplifier, optical gain media, optical fiber, infrared LEDs, lasers, and electroluminescent device, comprising a nanocrystal composition of the present invention. Preferably, the IR sensor or photodetector are modified for application as 3D cameras and 3D Time of flight cameras in mobile and consumer, automotive, medical, industrial, defence or aerospace applications. Preferably, the bio-imaging or bio-sensing compositions are modified for use as bio-labels or bio-tags in in vitro or ex vivo applications. Preferably, the infrared LEDs and electroluminescent devices are modified for use in telecommunication devices, night vision devices, solar energy conversion, surveillance devices, thermoelectric or energy generation applications.

### EXAMPLES

Examples are described hereunder illustrating the methods according to the present disclosure.

Whereas particular examples of this invention have been described below for purposes of illustration, it will be evident to those skilled in the art that numerous variations of the details of the present invention may be made without departing from the invention as defined in the appended claims.

Unless other indicated, all parts and all percentages in the following examples, as well as throughout the specification, are parts by weight or percentages by weight respectively.

Absorption spectra of colloidal quantum dots or quantum dots films were obtained on a JASCO V-770 UV-visible/NIR spectrometer which can provide measurements in the 400 to 3200 nm wavelength.

XRD data were collected on a Panalytical X′Pert PRO MPD diffractometer using Cu Ka1 X-radiation (I=1.5406 Å) at room temperature over a range of 10<2q<90°. In each case a few drops of the dispersed sample were placed on a glass microscope slide and allowed to evaporate. Data were analysed using Rigaku SmartLab Studio II software and the search and match carried out using the Crystallographic Open Database.

TEM images and high-resolution transmission electron microscope (HRTEM) images were obtained with an FEI Talos F200× microscope equipped with an X-FEG electron source. The experiment was performed using an acceleration voltage of 200 kV and a beam current of approximately 5 nA. Images were recorded with an FEI CETA 4k×4k CMOS camera. In each case a few drops of the dispersed quantum dots in solvent were placed on a carbon coated copper grid and allow to evaporate. Samples were used as such or treated with acetone then methanol to clean unwanted organic materials before imaging.

### Example 1—Preparation of Amorphous InAs Clusters

In a 3-neck 250 mL flask, 3.0 mmol of In(OAc) 3, 9.2 mmol of oleic acid and 15 mL of heptadecane were taken and evacuated under vacuum (~0.1 mbar) at 110° C. for 90 minutes. During this step, indium oleate was formed and 9 mmol of acetic acid was collected in the cold trap.

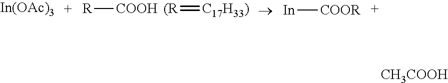

The flask was switched to nitrogen atmosphere and slowly cooled down to room temperature. The flask was transferred into a glovebox. In the glovebox, 0.96 mmol of tris(trimethylsilyl) arsine ((TMSi) 3As), 1.98 mmol dioctyl amine and 2.5 mL degassed heptadecane were taken in a 10 mL vial and vortexed thoroughly. Under constant stirring, indium oleate solution was mixed with TMSi—As solution to provide a composition comprising InAs clusters.

The composition comprising InAs clusters produced according to Example 1 was used as the composition comprising group III-V clusters in Example 2.

### Example 2—Preparation of Cuboctahedral InAs Nanocrystals with an Absorption Peak of Approximately 1400 nm

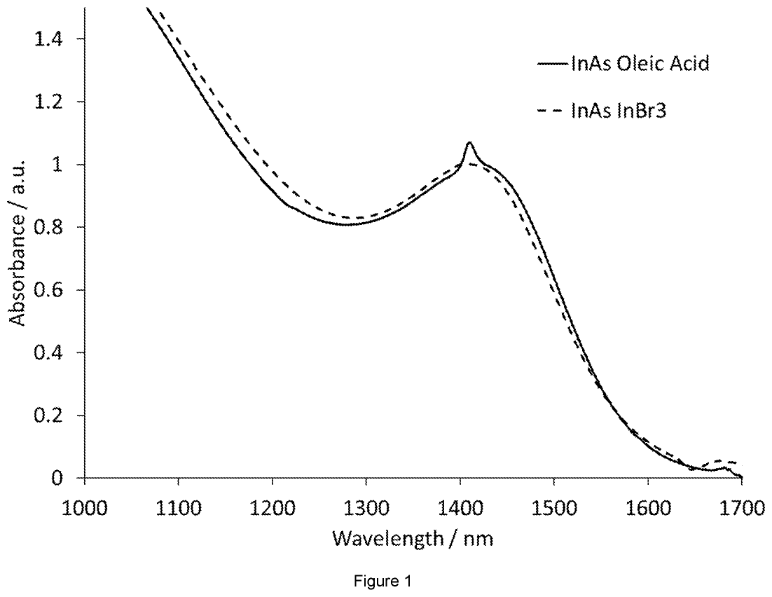

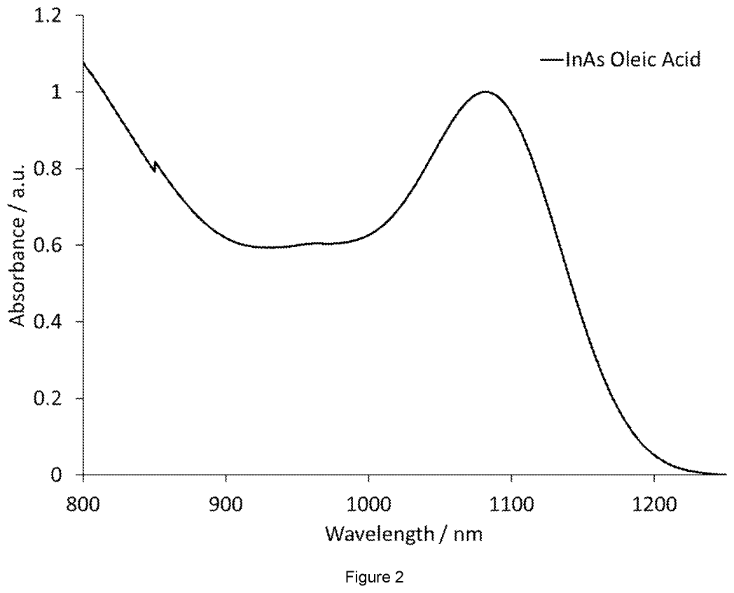

In this example, the preparation of InAs nanocrystals is split into two periods of crystal growth. First, InAs nanocrystals showing an absorption peak at around 1100 nm are formed (first growth time of 4.5 hours). The UV-vis spectra of these nanocrystals are shown in FIG. 2. Second, these nanocrystals are used to prepare nanocrystals showing an absorption peak at around 1400 nm (second growth time of 8 hours). The UV-vis spectra of these nanocrystals are shown in FIG. 1. The total growth time is 12.5 hours.

### 1100 nm QDs

In a 3-neck 250 ml flask, 0.4 mmol of In(OAc) 3, 1.2 mmol of oleic acid and 6 mL heptadecane were taken and degassed under vacuum (~0.1 mbar) at 110° C. for 90 minutes. During this step, Indium oleate was formed and 1.2 mmol of acetic acid was collected in the cold trap.

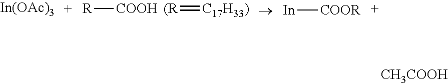

The flask was switched to nitrogen atmosphere and temperature was maintained at 100° C. In the glovebox, 0.32 mmol of (TMSi) 3As, 0.56 mmol of dioctyl amine and 1 mL of degassed heptadecane were loaded into a syringe. This solution is added to the indium oleate solution at 100° C. There was an immediate color change after the injection of arsenic precursor solution. The solution was simply heated up to 287° C.(*), once the temperature reached 287° C.(*), the growth was continued for 15 minutes. 10 mL of amorphous clusters solution of Example 1 was loaded into a syringe (diameter 20 mm) and added for nearly 4 h30 minutes at an injection rate of 2.230 mL/hour. After injecting all of the amorphous clusters solution, the solution was maintained at 287° C.(*) for another 10 minutes. While the cluster addition was taking place, 270 μL of dilute HBr solution in acetone (dilute HBr solution was prepared by diluting 40 μL of HBr in 2 mL acetone solution) was added over 4 h30 minutes time. The heating was turned off and the solution was cooled down naturally. CQDs were transferred to the glovebox for further use. CQDs growth was monitored by taking aliquots and measuring the absorption spectra.

The nanocrystals having an absorption maximum at approximately 1100 nm have an average size of 5.9±0.1 nm, with a standard deviation of 0.76 nm, as measured by TEM image analysis.

### 1400 nm QDs

In a 3-neck 250 ml flask connected under vacuum, 6 mL of InAs CQDs (50 mg/mL) (with an absorption max around 1100 nm) and 4 mL of degassed heptadecane was added. The flask contents were evacuated under vacuum (~0.1 mbar) at 100° C. for 30 minutes. The flask was switched to nitrogen. 200 μL of dilute HBr solution was added to the flask and maintained at 100° C. for 10 minutes. Then the temperature was increased to 285° C.(*). When temperature reaches around 285° C.(*), amorphous InAs clusters were loaded into a syringe and added using syringe pump at a rate of 1.16 mL/h for about 8 hours. While the cluster addition was taking place, 120 μL of dilute HBr was added over 8 hours. The heating was turned off and the solution was cooled down naturally. CQDs were transferred to the glovebox for further use. CQDs growth was monitored by taking aliquots and measuring the absorption spectra. The nanocrystals are cuboctahedral after this step.

### Purification

The flask contents were transferred to the glovebox. 5 mL of hexane was added to the QDs followed by 40 mL acetone. The solution was divided into centrifuge tubes and centrifuged at 6000 RPM for 10 minutes. The clear supernatant was discarded and the CQDs pellet was redispersed in hexanes. Acetone, IPA were added in 2:1 volume ratio and centrifuged the dots once again at 6000 RPM for 10 minutes. The supernatant was discarded and the CQDs pellet was redispersed in n-octane. The CQDs in octane were centrifuged at 6000 RPM for 10 minutes to remove any solid impurities. The sediment was discarded and clear QD solution is filtered through a 0.1 μm PTFE to yield the product.

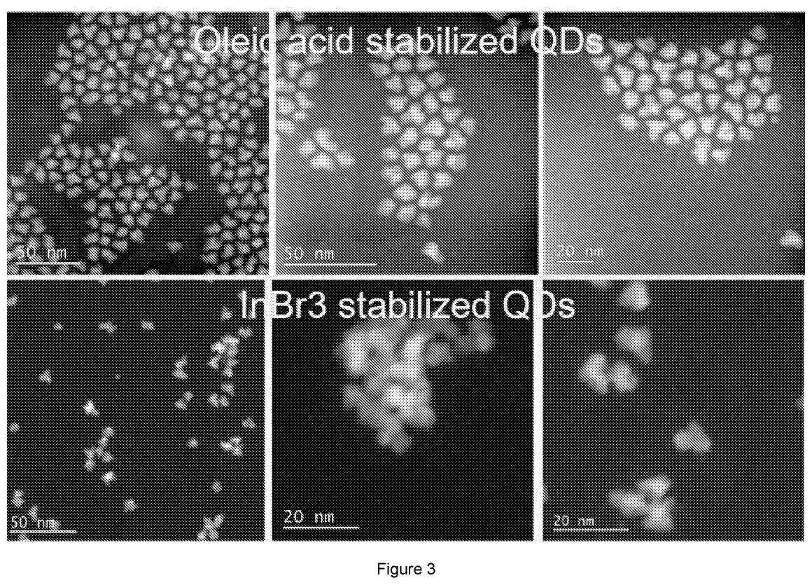

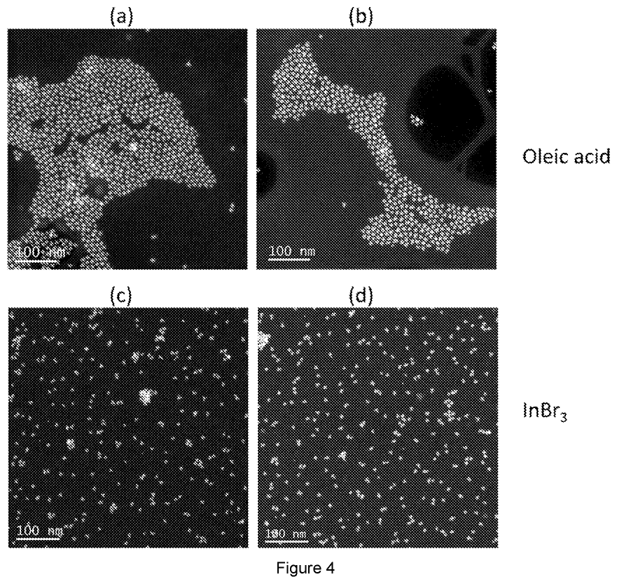

The resulting nanocrystals exhibit an absorption peak of approximately 1400 nm as shown in FIG. 1, and are cuboctahedral, as shown by the TEM images in FIGS. 3 and 4. After the inorganic ligand exchange, the dots come together to pack into a dense solid film which plays an important role in improved device performance.

Without wishing to be bound by theory, the improved facet alignment in the cuboctahedral nanocrystals leads to improved charge transfer.

The nanocrystals having an absorption maximum at approximately 1400 nm have an average size of 9.0±0.1 nm, with a standard deviation of 2.22 nm, as measured by TEM image analysis.

### Example 3—Preparation of Cuboctahedral InAs Nanocrystals with an Absorption Peak of Approximately 1600 nm

The method was the same as Example 2, except that it requires a second growth time of 14 hours and addition of the amorphous clusters solution at a rate of 1.1.6 mL/hour.

The nanocrystals are seen to be cuboctahedral in the TEM images.

### Example 4-Further Preparation of Cuboctahedral InAs Nanocrystals with an Absorption Peak of Approximately 1400 nm, Using 240° C. And a ZnBr₂ Additive

The method was the same as Example 2, and used the same process for producing the amorphous clusters as described in Example 1, with the following differences:
i) ZnBr₂ was included in the preparation of the clusters, and thus the first paragraph of the method of Example 1 was amended to the following: In a 3-neck 250 ml flask, 0.4 mmol of In(OAc) 3, 0.2 mmol of ZnBr₂, 1.2 mmol of oleic acid and 6 mL heptadecane were taken and degassed under vacuum (~0.1 mbar) at 110° C. for 90 minutes. During this step, Indium oleate was formed and 1.2 mmol of acetic acid was collected in the cold trap.
ii) a temperature of 240° C. was used to grow the nanocrystals instead of the growth temperatures marked with an asterisk (*) above.

The nanocrystals having an absorption maximum at approximately 1100 nm have an average size of 5.85±0.4 nm, as measured by TEM image analysis. The nanocrystals having an absorption maximum at approximately 1400 nm have an average size of 8.9±0.6 nm, as measured by TEM image analysis.

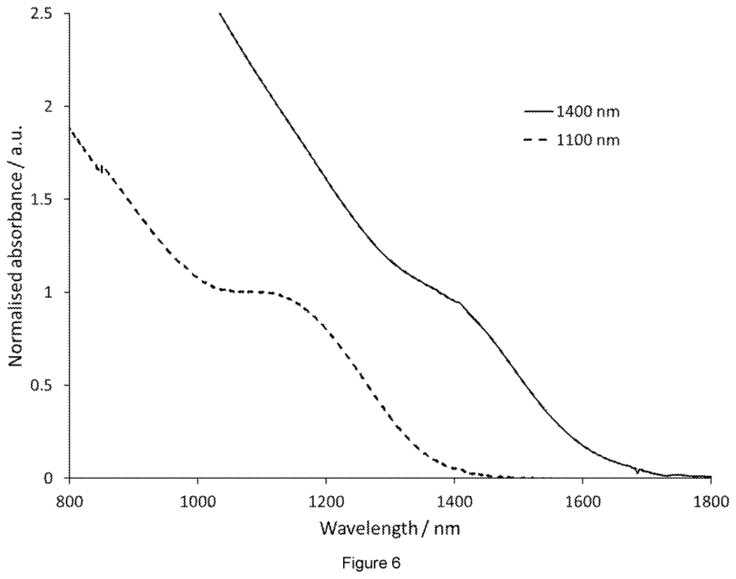

The absorption spectra of these nanocrystals are shown in FIG. 6.

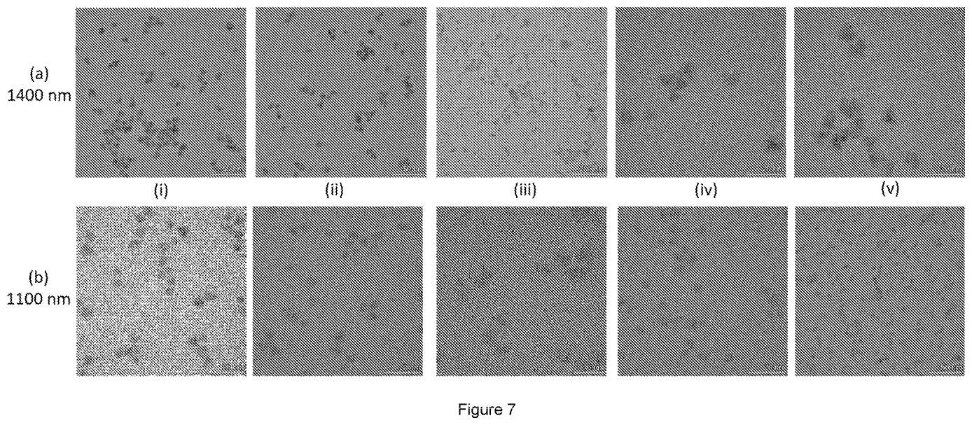

The nanocrystals are seen to be cuboctahedral in the TEM images in FIG. 7.

### EMBODIMENTS

1. A method for producing a group III-V nanocrystal composition, the method comprising:
   a) forming a first mixture comprising a first indium-containing compound and/or a first gallium-containing compound, a first ligand and a first solvent, wherein the first ligand is a C₆-C₂₄ organic compound comprising a functional group selected from the group consisting of amino, thiol, hydroxyl and carboxylic acid;
   b) adding at least one pnictogen-containing compound to the first mixture, and heating the first mixture to a growth temperature in the range of 200° C. to 350° C.;
   c) adding an oxide removal reagent and a composition comprising group III-V clusters to the first mixture at the growth temperature over a total growth time of at least 6 hours to provide the group III-V nanocrystal composition.
2. The method of embodiment 1, wherein the first mixture comprises the first indium-containing compound.
3. The method of embodiment 2, wherein the first indium-containing compound is selected from the group consisting of indium acetate, indium triformate, indium nitrate, indium nitrate hydrate, indium sulfate, indium hydroxide, indium trifluoromethanesulfonate, indium trifluoroacetylacetonate, indium hexafluoroacetylacetonate, indium acetylacetonate, indium trifluoroacetate, indium phosphate, indium perchlorate hydrate and mixtures thereof.
4. The method of embodiment 3, wherein the first indium-containing compound is indium acetate.
5. The method of any one of the preceding embodiments, wherein the first mixture comprises the first gallium-containing compound.
6. The method of embodiment 5, wherein the first gallium-containing compound is selected from the group consisting of gallium acetate, gallium nitrate, gallium sulfate, gallium hydroxide, gallium trifluoromethane sulfonate, gallium trifluoroacetylacetonate, gallium acetylacetonate, gallium trifluoroacetate, gallium phosphate, gallium perchlorate hydrate.
7. The method of embodiment 6, wherein the first gallium-containing compound is gallium acetate.
8. The method of any one of the preceding embodiments, wherein the first mixture comprises the first indium-containing compound and the first gallium-containing compound.
9. The method of any one of the preceding embodiments, wherein the first solvent is a non-polar solvent.
10. The method of any one of the preceding embodiments, wherein the first solvent has a relative permittivity at 20° C. of less than 3, more preferably less than 2.5.
11. The method of any one of the preceding embodiments, wherein the first solvent is selected from the group consisting of hydrocarbons of formula CₙH₂ₙ₊₂, alkylamines of formula CH₂ₙ₊₁NH₂, octadecene, oleylamine, octylamine, butylamine, dioctylamine, trioctylamine, heptadecane, hexadecane, oleic acid, tertiary phosphines and secondary phosphines, wherein n is in the range of 6 to 20.
12. The method of any one of the preceding embodiments, wherein the molar ratio of the at least one pnictogen-containing compound to the first indium-containing compound and the first gallium-containing compound is in the range of 0.5:1 to 1:0.5, preferably in the range of 0.5:1 to 1:1, preferably in the range of 0.6:1 to 1:1.
13. The method of any one of the preceding embodiments, wherein step b) comprises adding one pnictogen-containing compound, wherein the pnictogen-containing compound is selected from the group consisting of a phosphorus-containing compound, an arsenic-containing compound and an antimony-containing compound.
14. The method of any one of embodiments 1 to 12, wherein step b) comprises adding two pnictogen-containing compounds, wherein a first pnictogen-containing compound is an arsenic-containing compound, and wherein a second pnictogen-containing compound is a phosphorus-containing compound or an antimony-containing compound.
15. The method of embodiment 13 or embodiment 14, wherein the arsenic-containing compound is selected from the group consisting of an tris(trialkylsilyl) arsine, wherein the alkyl is methyl, ethyl, propyl, butyl or substituted, tris(trimethylgermyl) arsine, tris(triphenylsilyl) arsine, triphenyl arsine, aminoarsine (R₂N)₃As, wherein R is alkyl or aryl, arsenic halides, alkyl or aryl arsenous acids and mixtures thereof.
16. The method of embodiment 15, wherein the arsenic-containing compound is selected from the group consisting of tris(trimethylsilyl) arsine, tris(trimethylgermyl) arsine, tris(dimethylamino) arsine and mixtures thereof.
17. The method of any one of embodiments 13 to 16, wherein the antimony-containing compound is selected from the group consisting of tris(trialkylsilyl) antimony, wherein the alkyl is methyl, ethyl, propyl, butyl or substituted, tris(trimethylgermyl) antimony and aminoantimony (R₂N)₃Sb, wherein R is alkyl or aryl, antimony halides, antimony dialkaylamides, dialkylsilylamides, antimony hydrides, antimony aziridinides, antimony thiolates, antimony carbamates, antimony guanidinates and mixtures thereof.
18. The method of embodiment 17, wherein the antimony-containing compound is selected from the group consisting of tris(trimethylsilyl) antimony, tris(trimethylgermyl) antimony, tris(dimethylamino) antimony and mixtures thereof.
19. The method of embodiment 18, wherein the phosphorus-containing compound is selected from the group consisting of an tris(trialkylsilyl) phosphine, wherein the alkyl is methyl, ethyl, propyl, butyl or substituted, tris(trimethylgermyl) phosphine, tris(triphenylsilyl) phosphine, triphenyl phosphine, aminophosphine (R₂N)₃P, wherein R is alkyl or aryl, phosphonic halides, alkyl or aryl phosphonic acids and mixtures thereof, preferably wherein the phosphorus-containing compound is selected from the group consisting of tris(trimethylsilyl) phosphine, tris(trimethylgermyl) phosphine, tris(dimethylamino)phosphine and mixtures thereof, preferably wherein the phosphorus-containing compound is tris(trimethylsilyl) phosphine.
20. The method of any one of the preceding embodiments, wherein the molar ratio of the first ligand to the first indium-containing compound and/or gallium-containing compound is at least 2:1, preferably at least 2.5:1.
21. The method of any one of the preceding embodiments, wherein the first ligand is selected from the group consisting of alkylamines of formula CₙH₂ₙ₊₁NH₂, alkyl carboxylic acids of formula CₙH₂ₙ₊₁COOH, alkyl phosphonic acids of formula CₙH₂ₙ₊₁PO₃H₂, aromatic phosphonic acids, alkyl or aryl thiols, alkyl or aryl arsenous acids, and mixtures thereof, wherein n is in the range of 6 to 20.
22. The method of embodiment 21, wherein the first ligand is selected from the group consisting of aminobenzoic acids, dicarboxylic acids, aminoalkylcarboxylic acids, mercaptopropionic acid, mercaptobenzoic acid, thioalkanes, dithioalkanes, thiocarboxylic acids, thioglycolic acid, poly(ethylene glycol), poly(ethylene glycol) bis(3-aminopropyl) terminated, didodecyldimethylammonium bromide, n-dodecylammonium bromide, dodecyltrimethylammonium bromide, dimercaptosuccinic acid, oleic acid, oleylamine, bis(diphenylphosphino) methane and alkylamines.
23. The method of embodiment 22, wherein the first ligand is selected from the group consisting of mercaptopropionic acid, mercaptobenzoic acid, thioglycolic acid, poly(ethylene glycol), poly(ethylene glycol) bis(3-aminopropyl) terminated, didodecyldimethylammonium bromide, n-dodecylammonium bromide, dodecyltrimethylammonium bromide, dimercaptosuccinic acid, oleic acid, oleylamine, bis(diphenylphosphino) methane, preferably oleic acid, oleylamine, mercaptopropionic or mixtures thereof.
24. The method of any one of the preceding embodiments, wherein the growth temperature is in the range of 220° C. to 340° C., preferably in the range of 240° C. to 330° C., preferably in the range of 260° C. to 320° C., preferably in the range of 270° C. to 300° C. 25. The method of any one of the preceding embodiments, wherein the total growth time is in the range of 6 hours to 30 hours, preferably in the range of 8 hours to 26 hours, preferably in the range of 8 hours to 24 hours, preferably in the range of 9 hours to 23 hours, preferably in the range of 10 hours to 22 hours.
26. The method of embodiment 25, wherein the total growth time is in the range of 8 hours to 19 hours, preferably in the range of 9 hours to 18 hours, preferably in the range of 10 hours to 17 hours, preferably in the range of 11 hours to 16 hours.
27. The method of embodiment 25, wherein the total growth time is in the range of 14 hours to 28 hours, preferably in the range of 15 hours to 26 hours, preferably in the range of 16 hours to 24 hours, preferably in the range of 17 hours to 22 hours.
28. The method of any one of the preceding embodiments, wherein the nanocrystal composition comprises group III-V nanocrystals, wherein the nanocrystals comprise a crystalline core and a plurality of first ligands coordinated to a surface of the crystalline core.
29. The method of embodiment 28, wherein the oxide removal reagent is a compound capable of reacting with oxides on the surface of the crystalline core to remove the oxides from the surface of the crystalline core.
30 The method of any one of the preceding embodiments, wherein the oxide removal reagent is selected from the group consisting of hydrochloric acid, hydrobromic acid, hydroiodic acid, hydrofluoric acid, hypo phosphorus acid, phosphoric acid, ammonium hydroxide, potassium hydroxide, sodium hydroxide, zinc halides, indium halides and metal halides, wherein the metal is bismuth, lead or cadmium.
31. The method of embodiment 30, wherein the oxide removal reagent is hydrobromic acid.
32. The method of any one of the preceding embodiments, wherein the molar ratio of the oxide removal reagent to the sum of the first indium-containing compound and/or first gallium-containing compound is in the range of 1:100 to 1:1,000,000, preferably in the range of 1:1000 to 1:100,000, more preferably in the range of 1:000 to 1:50,000.
33. The method of any one of the preceding embodiments, wherein the concentration of oxide removal reagent in the first mixture is in the range of 5 μM to 1000 UM, preferably in the range of 50 UM to 500 μM.
34. The method of any one of the preceding embodiments, wherein steps a), b) and c) take place under inert conditions.
35. The method of any one of the preceding embodiments, wherein step a) further comprises heating the first mixture to a first temperature in the range of 50° C. to 150° C.
36. The method of embodiment 35, wherein the first temperature is in the range of 60° C. to 140° C., preferably in the range of 70° C. to 130° C., preferably in the range of 80° C. to 120° C., preferably in the range of 90° C. to 110° C.
37. The method of any one of the preceding embodiments, wherein step c) comprises:
   i) adding a first oxide removal reagent and the composition comprising group III-V clusters to the first mixture at the growth temperature over a first growth time in the range of 2 hours to 8 hours to provide a plurality of nanocrystals in the first mixture;
   ii) allowing the first mixture to cool;
   iii) adding a second oxide removal reagent to the first mixture, and heating the first mixture to the growth temperature;
   iv) adding a third oxide removal reagent and the composition comprising group III-V clusters to the first mixture over a second growth time in the range of 6 hours to 20 hours at the growth temperature to provide the group III-V nanocrystal composition.
38. The method of embodiment 37, wherein step iii) further comprises adding a second oxide removal reagent to the first mixture before heating the first mixture to the growth temperature.
39. The method of embodiment 37 or embodiment 38, wherein step i) further comprises heating the first mixture to a third temperature in the range of 50° C. to 150° C., preferably the range of 60° C. to 140° C., preferably the range of 70° C. to 130° C., preferably the range of 80° C. to 120° C., preferably the range of 90° C. to 110° C.
40. The method of any one of embodiments 37 to 39, wherein the first growth time is in the range of 2.5 hours to 7 hours, preferably in the range of 3 hours to 6 hours, preferably in the range of 3.5 hours to 5.5 hours, preferably in the range of 4 hours to 5 hours.
41. The method of any one of embodiments 37 to 40, wherein the second growth time is in the range of 6 hours to 18 hours, preferably in the range of 6.5 hours to 16 hours, preferably in the range of 7 hours to 15 hours.
42. The method of any one of embodiments 37 to 41, wherein the nanocrystal composition comprises group III-V nanocrystals, wherein the nanocrystals comprise a crystalline core and a plurality of first ligands coordinated to a surface of the crystalline core, and wherein the first oxide removal reagent, the second oxide removal reagent and the third oxide removal reagent are each independently a compound capable of reacting with oxides on the surface of the crystalline core to remove the oxides from the surface of the crystalline core.
43. The method of any one of embodiments 37 to 42, wherein the first oxide removal reagent, the second oxide removal reagent and the third oxide removal reagent are each independently are a compound selected from the group consisting of hydrochloric acid, hydrobromic acid, hydroiodic acid, hydrofluoric acid, hypo phosphorus acid, phosphoric acid, ammonium hydroxide, potassium hydroxide, sodium hydroxide, zinc halides, indium halides and metal halides, wherein the metal is bismuth, lead or cadmium.
44. The method of any one of embodiments 37 to 43, wherein the first oxide removal reagent, the second oxide removal reagent and the third oxide removal reagent are the same compound.
45. The method of any one of embodiments 37 to 44, wherein the first oxide removal reagent, the second oxide removal reagent and the third oxide removal reagent are hydrobromic acid.
46. The method of any one of the preceding embodiments, wherein the first mixture comprises a compound selected from the group consisting of secondary amines, zinc carboxylates, and mixtures thereof, preferably wherein the compound is dioctyl amine.
47. The method of any one of the preceding embodiments, wherein the group III-V clusters are amorphous particles comprising a group III element and a group V element, wherein the particles have a diameter of less than 2 nm.
48. The method of embodiment 48, wherein the group III-V clusters comprise phosphorus, arsenic, antimony or mixtures thereof.
49. The method of embodiment 48 or embodiment 49, wherein the group III-V clusters are amorphous clusters exhibiting an absorption peak in the range of 900 nm to 1200 nm, preferably in the range of 1000 nm to 1200 nm.
50. The method of any one of the preceding embodiments, wherein the method further comprises preparing the composition comprising group III-V clusters, comprising:
   A) adding a second indium-containing compound and/or a second gallium-containing compound, and a second ligand to a second solvent to form a second mixture, wherein the second ligand is a C₆-C₂₄ organic compound comprising a functional group selected from the group consisting of amino, thiol, hydroxyl and carboxylic acid;
   B) adding at least one pnictogen-containing compound to the second mixture under constant agitation to provide the group III-V clusters.
51. The method of embodiment 50, wherein the second mixture comprises the second indium-containing compound.
52. The method of embodiment 51, wherein the second indium-containing compound is selected from the group consisting of indium acetate, indium triformate, indium nitrate, indium nitrate hydrate, indium sulfate, indium hydroxide, indium trifluoromethanesulfonate, indium trifluoroacetylacetonate, indium hexafluoroacetylacetonate, indium acetylacetonate, indium trifluoroacetate, indium phosphate, indium perchlorate hydrate and mixtures thereof.
53. The method of embodiment 52, wherein the second indium-containing compound is indium acetate.
54. The method of any one of embodiments 50 to 53, wherein the second mixture comprises the second gallium-containing compound.
55. The method of embodiment 54, wherein the second gallium-containing compound is selected from the group consisting of gallium acetate, gallium nitrate, gallium sulfate, gallium hydroxide, gallium trifluoromethane sulfonate, gallium trifluoroacetylacetonate, gallium acetylacetonate, gallium trifluoroacetate, gallium phosphate, gallium perchlorate hydrate.
56. The method of embodiment 55, wherein the second gallium-containing compound is gallium acetate.
57 The method of any one of embodiments 50 to 56, wherein the second mixture comprises the second indium-containing compound and the second gallium-containing compound.
58. The method of any one of embodiments 50 to 57, wherein the second solvent is a non-polar solvent.
59. The method of any one of embodiments 50 to 58, wherein the second solvent has a relative permittivity at 20° C. of less than 3, more preferably less than 2.5.
60. The method of any one of embodiments 50 to 59, wherein the second solvent is selected from the group consisting of hydrocarbons of formula CₙH₂ₙ₊₂, alkylamines of formula CₙH₂ₙ₊₁NH₂, octadecene, oleylamine, octylamine, butylamine, dioctylamine, trioctylamine, heptadecane, hexadecane, oleic acid, tertiary phosphines and secondary phosphines, wherein n is in the range of 6 to 20.
61. The method of any one of embodiments 50 to 60, wherein the wherein the second solvent is the same as the first solvent.
62. The method of any one of embodiments 50 to 61, wherein the molar ratio of the at least one pnictogen-containing compound to the second indium-containing compound and the second gallium-containing compound is in the range of 0.5:1 to 1:0.5, preferably in the range of 0.5:1 to 1:1, preferably in the range of 0.6:1 to 1:1.
63. The method of any one of embodiments 50 to 62, wherein step B) comprises adding one pnictogen-containing compound, wherein the pnictogen-containing compound is selected from the group consisting of a phosphorus-containing compound, an arsenic-containing compound and an antimony-containing compound.
64. The method of any one of embodiments 50 to 62, wherein step B) comprises adding two pnictogen-containing compounds, wherein a first pnictogen-containing compound is an arsenic-containing compound, and wherein a second pnictogen-containing compound is a phosphorus-containing compound or an antimony-containing compound.
65. The method of embodiment 63 or embodiment 64, wherein the arsenic-containing compound is selected from the group consisting of an tris(trialkylsilyl) arsine, wherein the alkyl is methyl, ethyl, propyl, butyl or substituted, tris(trimethylgermyl) arsine, tris(triphenylsilyl) arsine, triphenyl arsine, aminoarsine (R₂N)₃As, wherein R is alkyl or aryl, arsenic halides, alkyl or aryl arsenous acids and mixtures thereof.
66 The method of embodiment 65, wherein the arsenic-containing compound is tris(trimethylsilyl) arsine.
67. The method of any one of embodiments 63 to 66, wherein the antimony-containing compound is selected from the group consisting of tris(trialkylsilyl) antimony, wherein the alkyl is methyl, ethyl, propyl, butyl or substituted, tris(trimethylgermyl) antimony and aminoantimony (R₂N)₃Sb, wherein R is alkyl or aryl, antimony halides, antimony dialkaylamides, dialkylsilylamides, antimony hydrides, antimony aziridinides, antimony thiolates, antimony carbamates and antimony guanidinates.
68. The method of embodiment 67, wherein the antimony-containing compound is tris(trimethylsilyl) antimony.
69. The method of any one of embodiments 63 to 68, wherein the phosphorus-containing compound is selected from the group consisting of . . .
70. The method of any one of embodiments 50 to 69, wherein the molar ratio of the second ligand to the second indium-containing compound is at least 2:1, preferably at least 2.5:1.
71. The method of any one of embodiments 50 to 70, wherein the second ligand is selected from the group consisting of alkylamines of formula CₙH₂ₙ₊₁NH₂, alkyl carboxylic acids of formula CₙH₂ₙ₊₁COOH, alkyl phosphonic acids of formula CₙH₂ₙ₊₁PO₃H₂, aromatic phosphonic acids, alkyl or aryl thiols, alkyl or aryl arsenous acids, and mixtures thereof, wherein n is in the range of 6 to 20.
72. The method of embodiment 71, wherein the second ligand is selected from the group consisting of aminobenzoic acids, dicarboxylic acids, aminoalkylcarboxylic acids, mercaptopropionic acid, mercaptobenzoic acid, thioalkanes, dithioalkanes, thiocarboxylic acids, thioglycolic acid, poly(ethylene glycol), poly(ethylene glycol) bis(3-aminopropyl) terminated, didodecyldimethylammonium bromide, n-dodecylammonium bromide, dodecyltrimethylammonium bromide, dimercaptosuccinic acid, oleic acid, oleylamine, bis(diphenylphosphino) methane and alkylamines.
73. The method of embodiment 72, wherein the second ligand is selected from the group consisting of mercaptopropionic acid, mercaptobenzoic acid, thioglycolic acid, poly(ethylene glycol), poly(ethylene glycol) bis(3-aminopropyl) terminated, didodecyldimethylammonium bromide, n-dodecylammonium bromide, dodecyltrimethylammonium bromide, dimercaptosuccinic acid, oleic acid, oleylamine, bis(diphenylphosphino) methane.
74. The method of any one of embodiments 50 to 73, wherein the second ligand is the same as the first ligand.
75. A group III-V nanocrystal composition obtainable by any one of the preceding embodiments.
76. A substantially cuboctahedral group III-V nanocrystal.
77. The nanocrystal of embodiment 76, wherein the nanocrystal is cuboctahedral.
78. The nanocrystal of embodiment 76 or embodiment 77, wherein the nanocrystal comprises a crystalline core comprising indium and/or gallium, and a pnictogen.
79. The nanocrystal of embodiment 78, wherein the pnictogen is phosphorus, arsenic, antimony, or a mixture thereof.
80. The nanocrystal of embodiment 79, wherein the pnictogen is arsenic, antimony, or a mixture thereof.
81. The nanocrystal of any one of embodiments 78 to 80, wherein a plurality of ligands are coordinated to a surface of the crystalline core.
82. The nanocrystal of embodiment 81, wherein the ligands are organic compounds, inorganic compounds, or a mixture thereof.
83. The nanocrystal of any one of embodiments 76 to 82, wherein the nanocrystal composition exhibits an absorption peak in the range of 500 nm to 3000 nm, preferably in the range of 1300 nm to 1700 nm, preferably in the range of 1320 nm to 1680 nm, preferably in the range of 1340 nm to 1660 nm, preferably in the range of 1360 nm to 1640 nm, preferably in the range of 1380 nm to 1620 nm, preferably in the range of 1400 nm to 1600 nm.
84. The nanocrystal of any one of embodiments 76 to 83, wherein the nanocrystal composition exhibits an emission peak in the range of 1300 nm to 1900 nm, preferably in the range of 1320 nm to 1880 nm, preferably in the range of 1340 nm to 1860 nm, preferably in the range of 1360 nm to 1840 nm, preferably in the range of 1380 nm to 1820 nm, preferably in the range of 1400 nm to 1800 nm.
85. A group III-V nanocrystal composition comprising a plurality of nanocrystals according to any one of embodiments 76 to 84.
86. The nanocrystal composition of embodiment 85, wherein the nanocrystals have a median particle diameter in the range of 2 nm to 20 nm, preferably 2.5 nm to 15 nm, preferably 3 nm to 12 nm, preferably 4 nm to 10 nm.
87. The nanocrystal composition of embodiment 85 or embodiment 86, wherein the nanocrystal composition exhibits absorption full width at half maximum (FWHM) values of less than 250 nm, preferably less than 225 nm, preferably less than 200 nm, preferably less than 175 nm.
88. The nanocrystal composition of any one of embodiments 85 to 87, wherein the nanocrystal composition exhibits emission full width at half maximum (FWHM) values of less than 250 nm, preferably less than 225 nm, preferably less than 200 nm, preferably less than 175 nm.
89. The nanocrystal composition of any one of embodiments 85 to 88, wherein the nanocrystal composition exhibits a peak to valley ratio in the range of 1 to 4, preferably in the range of 1 to 3.5, preferably in the range of 1 to 3, preferably in the range of 1.1 to 2.5, preferably in the range of 1.2 to 2.5.
90. The nanocrystal composition of any one of embodiments 85 to 89, wherein the nanocrystals exhibit a relative size dispersion of less than 25%, preferably less than 15%, preferably less than 10%, preferably in the range of 1% to 25%, preferably in the range of 1% to 20%, preferably in the range of 2% to 15%, preferably in the range of 3% to 10%.
91. An ink comprising the group III-V nanocrystal composition of any one of embodiments 85 to 90.
92. A film comprising the group III-V nanocrystal composition of any one of embodiments 85 to 90.

### REFERENCES

1. Alivisatos et al. Appl. Phys. Lett. 1996, 69, 1432.
2. Bawendi et al. Nat. Comms. 2016, 7, 12749).
3. Jeong et al. Nat. Comms. 2018, 9, 1.
4. Sargent et al Nano. Lett., 2021, 21, 6057.
5. Talapin et al. Chem. Mater. 2016, 28, 6797.
6. Bawendi et al. J. Am. Chem. Soc., 2020, 142, 4088.
7. Hens et al. J. Am. Chem. Soc. 2021, 143, 4290.
8 Pietryga et al J. Am. Chem. Soc. 2008, 130, 14, 4879-4885.
9. Lee et al Adv. Mater. 2003, 15 (5), 441.
10. Biondi et al Adv. Mater. 2021, 2101056.
11. Chaudret et al. Journal of American Chemical Society, 2010, 18147-18157, 132 (51).

1. A method for producing a group III-V nanocrystal composition, the method comprising:
a) forming a first mixture comprising a first indium-containing compound and/or a first gallium-containing compound, a first ligand and a first solvent, wherein the first ligand is a C₆-C₂₄ organic compound comprising a functional group selected from the group consisting of amino, thiol, hydroxyl and carboxylic acid;
b) adding at least one pnictogen-containing compound to the first mixture, and heating the first mixture to a growth temperature in the range of 200° C. to 350° C.;
c) adding an oxide removal reagent and a composition comprising group III-V clusters to the first mixture at the growth temperature over a total growth time in the range of at least 6 hours to provide the group III-V nanocrystal composition.

2. The method of claim 1, wherein the first mixture comprises the first indium-containing compound,
preferably wherein the first indium-containing compound is selected from the group consisting of indium acetate, indium triformate, indium nitrate, indium nitrate hydrate, indium sulfate, indium hydroxide, indium trifluoromethanesulfonate, indium trifluoroacetylacetonate, indium hexafluoroacetylacetonate, indium acetylacetonate, indium trifluoroacetate, indium phosphate, indium perchlorate hydrate and mixtures thereof,
preferably wherein the first indium-containing compound is indium acetate.

3. The method of claim 1, wherein the first mixture comprises the first gallium-containing compound,
preferably wherein the first gallium-containing compound is selected from the group consisting of gallium acetate, gallium nitrate, gallium sulfate, gallium hydroxide, gallium trifluoromethane sulfonate, gallium trifluoroacetylacetonate, gallium acetylacetonate, gallium trifluoroacetate, gallium phosphate, gallium perchlorate hydrate,
preferably wherein the first gallium-containing compound is gallium acetate.

4. The method of claim 1, wherein the first solvent is a non-polar solvent,
preferably wherein the first solvent has a relative permittivity at 20° C. of less than 3, more preferably less than 2.5,
preferably wherein the first solvent is selected from the group consisting of hydrocarbons of formula CₙH₂ₙ₊₂, alkylamines of formula CₙH₂ₙ₊₁NH₂, octadecene, oleylamine, octylamine, butylamine, dioctylamine, trioctylamine, heptadecane, hexadecane, oleic acid, tertiary phosphines and secondary phosphines, wherein n is in the range of 6 to 20.

5. The method of claim 1, wherein the molar ratio of the at least one pnictogen-containing compound to the first indium-containing compound and the first gallium-containing compound is in the range of 0.5:1 to 1:0.5, preferably in the range of 0.5:1 to 1:1, preferably in the range of 0.6:1 to 1:1.

6. The method of claim 1, wherein step b) comprises adding one pnictogen-containing compound, wherein the pnictogen-containing compound is selected from the group consisting of a phosphorus-containing compound, an arsenic-containing compound and an antimony-containing compound, or
wherein step b) comprises adding two pnictogen-containing compounds, wherein a first pnictogen-containing compound is an arsenic-containing compound, and wherein a second pnictogen-containing compound is a phosphorus-containing compound or an antimony-containing compound.

7. The method of claim 6, wherein the arsenic-containing compound is selected from the group consisting of an tris(trialkylsilyl) arsine, wherein the alkyl is methyl, ethyl, propyl, butyl or substituted, tris(trimethylgermyl) arsine, tris(triphenylsilyl) arsine, triphenyl arsine, aminoarsine (R₂N)₃As, wherein R is alkyl or aryl, arsenic halides, alkyl or aryl arsenous acids and mixtures thereof,
preferably wherein the arsenic-containing compound is selected from the group consisting of tris(trimethylsilyl) arsine, tris(trimethylgermyl) arsine, tris(dimethylamino) arsine and mixtures thereof.

8. The method of claim 6, wherein the antimony-containing compound is selected from the group consisting of tris(trialkylsilyl) antimony, wherein the alkyl is methyl, ethyl, propyl, butyl or substituted, tris(trimethylgermyl) antimony and aminoantimony (R₂N)₃Sb, wherein R is alkyl or aryl, antimony halides, antimony dialkaylamides, dialkylsilylamides, antimony hydrides, antimony aziridinides, antimony thiolates, antimony carbamates, antimony guanidinates and mixtures thereof, preferably wherein the antimony-containing compound is selected from the group consisting of tris(trimethylsilyl) antimony, tris(trimethylgermyl) antimony, tris(dimethylamino) antimony and mixtures thereof.

9. The method of claim 6, wherein the phosphorus-containing compound is selected from the group consisting of an tris(trialkylsilyl) phosphine, wherein the alkyl is methyl, ethyl, propyl, butyl or substituted, tris(trimethylgermyl) phosphine, tris(triphenylsilyl) phosphine, triphenyl phosphine, aminophosphine (R₂N)₃P, wherein R is alkyl or aryl, phosphonic halides, alkyl or aryl phosphonic acids and mixtures thereof, preferably wherein the phosphorus-containing compound is selected from the group consisting of tris(trimethylsilyl) phosphine, tris(trimethylgermyl) phosphine, tris(dimethylamino)phosphine and mixtures thereof, preferably wherein the phosphorus-containing compound is tris(trimethylsilyl) phosphine.

10. The method of claim 1, wherein the molar ratio of the first ligand to the first indium-containing compound and/or gallium-containing compound is at least 2:1, preferably at least 2.5:1.

11. The method of claim 1, wherein the first ligand is selected from the group consisting of alkylamines of formula CₙH₂ₙ₊₁NH₂, alkyl carboxylic acids of formula CₙH₂ₙ₊₁COOH, alkyl phosphonic acids of formula CₙH₂ₙ₊₁PO₃H₂, aromatic phosphonic acids, alkyl or aryl thiols, alkyl or aryl arsenous acids, and mixtures thereof, wherein n is in the range of 6 to 20,
preferably wherein the first ligand is selected from the group consisting of aminobenzoic acids, dicarboxylic acids, aminoalkylcarboxylic acids, mercaptopropionic acid, mercaptobenzoic acid, thioalkanes, dithioalkanes, thiocarboxylic acids, thioglycolic acid, poly(ethylene glycol), poly(ethylene glycol) bis(3-aminopropyl) terminated, didodecyldimethylammonium bromide, n-dodecylammonium bromide, dodecyltrimethylammonium bromide, dimercaptosuccinic acid, oleic acid, oleylamine, bis(diphenylphosphino) methane and alkylamines,
preferably wherein the first ligand is selected from the group consisting of mercaptopropionic acid, mercaptobenzoic acid, thioglycolic acid, poly(ethylene glycol), poly(ethylene glycol) bis(3-aminopropyl) terminated, didodecyldimethylammonium bromide, n-dodecylammonium bromide, dodecyltrimethylammonium bromide, dimercaptosuccinic acid, oleic acid, oleylamine, bis(diphenylphosphino) methane, preferably oleic acid, oleylamine, mercaptopropionic or mixtures thereof.

12. The method of claim 1, wherein the growth temperature is in the range of 220° C. to 340° C., preferably in the range of 240° C. to 330° C., preferably in the range of 260° C. to 320° C., preferably in the range of 270° C. to 300° C.

13. The method of claim 1, wherein the total growth time is in the range of 6 hours to 30 hours, preferably in the range of 8 hours to 26 hours, preferably in the range of 8 hours to 24 hours, preferably in the range of 9 hours to 23 hours, preferably in the range of 10 hours to 22 hours,
optionally wherein the total growth time is in the range of 8 hours to 19 hours, preferably in the range of 9 hours to 18 hours, preferably in the range of 10 hours to 17 hours, preferably in the range of 11 hours to 16 hours, or
optionally wherein the total growth time is in the range of 14 hours to 28 hours, preferably in the range of 15 hours to 26 hours, preferably in the range of 16 hours to 24 hours, preferably in the range of 17 hours to 22 hours.

14. The method of claim 1, wherein the nanocrystal composition comprises group III-V nanocrystals, wherein the nanocrystals comprise a crystalline core and a plurality of first ligands coordinated to a surface of the crystalline core,
preferably wherein the oxide removal reagent is a compound capable of reacting with oxides on the surface of the crystalline core to remove the oxides from the surface of the crystalline core,
preferably wherein the oxide removal reagent is selected from the group consisting of hydrochloric acid, hydrobromic acid, hydroiodic acid, hydrofluoric acid, hypo phosphorus acid, phosphoric acid, ammonium hydroxide, potassium hydroxide, sodium hydroxide, zinc halides, indium halides and metal halides, wherein the metal is bismuth, lead or cadmium,
preferably wherein the oxide removal reagent is hydrobromic acid.

15. The method of claim 1, wherein the molar ratio of the oxide removal reagent to the sum of the first indium-containing compound and/or first gallium-containing compound is in the range of 1:100 to 1:1,000,000, preferably in the range of 1:1000 to 1:100,000, more preferably in the range of 1:000 to 1:50,000, and/or
wherein the concentration of oxide removal reagent in the first mixture is in the range of 5 μM to 1000 μM, preferably in the range of 50 μM to 500 μM.

16. The method of claim 1, wherein step a) further comprises heating the first mixture to a first temperature in the range of 50° C. to 150° C.,
preferably wherein the first temperature is in the range of 60° C. to 140° C., preferably in the range of 70° C. to 130° C., preferably in the range of 80° C. to 120° C., preferably in the range of 90° C. to 110° C.

17. The method of claim 1, wherein step c) comprises:
i) adding a first oxide removal reagent and the composition comprising group III-V clusters to the first mixture at the growth temperature over a first growth time in the range of 2 hours to 8 hours to provide a plurality of nanocrystals in the first mixture;
ii) allowing the first mixture to cool;
iii) heating the first mixture to the growth temperature;
iv) adding a third oxide removal reagent and the composition comprising group III-V clusters to the first mixture over a second growth time in the range of 6 hours to 20 hours at the growth temperature to provide the group III-V nanocrystal composition,
preferably wherein step iii) further comprises adding a second oxide removal reagent to the first mixture before heating the first mixture to the growth temperature,
preferably wherein step i) further comprises heating the first mixture to a third temperature in the range of 50° C. to 150° C., preferably the range of 60° C. to 140° C., preferably the range of 70° C. to 130° C., preferably the range of 80° C. to 120° C., preferably the range of 90° C. to 110° C.

18. The method of claim 17, wherein the first growth time is in the range of 2.5 hours to 7 hours, preferably in the range of 3 hours to 6 hours, preferably in the range of 3.5 hours to 5.5 hours, preferably in the range of 4 hours to 5 hours, and/or
wherein the second growth time is in the range of 6 hours to 18 hours, preferably in the range of 6.5 hours to 16 hours, preferably in the range of 7 hours to 15 hours.

19. The method of claim 17, wherein the first oxide removal reagent, the second oxide removal reagent and the third oxide removal reagent are the same compound,
preferably wherein the first oxide removal reagent, the second oxide removal reagent and the third oxide removal reagent are hydrobromic acid.

20. The method of claim 1, wherein the group III-V clusters are amorphous particles comprising a group III element and a group V element, wherein the particles have a diameter of less than 2 nm,
preferably wherein the group III-V clusters comprise phosphorus, arsenic, antimony or mixtures thereof.

21. A group III-V nanocrystal composition obtainable by claim 1.

22. A substantially cuboctahedral group III-V nanocrystal.

23. The nanocrystal of claim 22, wherein the nanocrystal comprises a crystalline core comprising indium and/or gallium, and a pnictogen,
preferably wherein the pnictogen is phosphorus, arsenic, antimony, or a mixture thereof,
preferably wherein the pnictogen is arsenic, antimony, or a mixture thereof.

24. The nanocrystal of claim 22, wherein a plurality of ligands are coordinated to a surface of the crystalline core,
preferably wherein the ligands are organic compounds, inorganic compounds, or a mixture thereof.

25. The nanocrystal of claim 22, wherein the nanocrystal composition exhibits an absorption peak in the range of 500 nm to 3000 nm, preferably in the range of 1300 nm to 1700 nm, preferably in the range of 1320 nm to 1680 nm, preferably in the range of 1340 nm to 1660 nm, preferably in the range of 1360 nm to 1640 nm, preferably in the range of 1380 nm to 1620 nm, preferably in the range of 1400 nm to 1600 nm, and/or
wherein the nanocrystal composition exhibits an emission peak in the range of 1300 nm to 1900 nm, preferably in the range of 1320 nm to 1880 nm, preferably in the range of 1340 nm to 1860 nm, preferably in the range of 1360 nm to 1840 nm, preferably in the range of 1380 nm to 1820 nm, preferably in the range of 1400 nm to 1800 nm.

26. A group III-V nanocrystal composition comprising a plurality of nanocrystals according to claim 22.

27. The nanocrystal composition of claim 26, wherein the nanocrystal composition exhibits absorption full width at half maximum (FWHM) values of less than 250 nm, preferably less than 225 nm, preferably less than 200 nm, preferably less than 175 nm, and/or
wherein the nanocrystal composition exhibits emission full width at half maximum (FWHM) values of less than 250 nm, preferably less than 225 nm, preferably less than 200 nm, preferably less than 175 nm.

28. The nanocrystal composition of claim 26, wherein the nanocrystal composition exhibits a peak to valley ratio in the range of 1 to 4, preferably in the range of 1 to 3.5, preferably in the range of 1 to 3, preferably in the range of 1.1 to 2.5, preferably in the range of 1.2 to 2.5, and/or
wherein the nanocrystals exhibit a relative size dispersion of less than 25%, preferably less than 15%, preferably less than 10%, preferably in the range of 1% to 25%, preferably in the range of 1% to 20%, preferably in the range of 2% to 15%, preferably in the range of 3% to 10%.

29. An ink or a film comprising the group III-V nanocrystal composition of claim 26.
